(19) United States
(12) Patent Application Publication
(10) Pub. No.: US 2026/0243752 A1
(43) Pub. Date: Aug. 20, 2026

(54) DATA PROCESSING APPARATUS AND DATA PROCESSING METHOD

(71) Applicant: SONY GROUP CORPORATION, TOKYO (JP)

(72) Inventor: SHO MURAKOSHI, KANAGAWA (JP)

(21) Appl. No.: 19/161,285

(22) PCT Filed: Feb. 29, 2024
(86) PCT No.: PCT/JP2024/007493

§ 371 Date: Sep. 1, 2025

(30) Foreign Application Priority Data
Mar. 24, 2023 (JP) 2023-048813

(51) Int. Cl. G01N 33/24

(52) U.S. Cl. CPC G01N 33/246; G01N 33/245

(57) ABSTRACT

The present technology relates to a data processing apparatus and a data processing method enabling appropriate detection of a moisture amount of a field to appropriately perform irrigation control. There are provided a first creation unit configured to create a relative moisture amount map regarding a relative moisture amount in a field, an acquisition unit configured to acquire moisture amount data from a moisture amount sensor installed in the field, and a second creation unit configured to convert the relative moisture amount into an absolute moisture amount by using the moisture amount data, to create an absolute moisture amount map. The present technology can be applied to, for example, a data processing apparatus that acquires data for controlling irrigation of a field, appropriately detects a moisture amount in the field, and controls the irrigation.

### TECHNICAL FIELD

The present technology relates to a data processing apparatus and a data processing method, and for example, relates to a data processing apparatus and a data processing method capable of appropriately estimating a moisture amount of a field to perform processing according to the moisture amount.

### BACKGROUND ART

A moisture amount of soil is one of factors that affect a harvest amount of crops. In order to increase a harvest amount of crops, it is desired to maintain a moisture amount in soil suitable for a type, a growth stage, and the like of the crops. It has been proposed to present information about a moisture amount in soil to a user (see, for example, Patent Documents 1 and 2).

### CITATION LIST

### Patent Document

Patent Document 1: US Patent Application Publication No. 2016/0157446
Patent Document 2: US Patent Application Publication No. 2018/0146631

### SUMMARY OF THE INVENTION

### Problems to be Solved by the Invention

A moisture amount in a field may not be uniform. In particular, in a vast field, it has been difficult to keep the moisture amount of soil uniform. It is also difficult to perform irrigation control for keeping the moisture amount uniform, and there has been a possibility that the moisture amount in the field becomes non-uniform, and the harvest amount of crops is affected.

It is desired to grasp variations in a moisture amount in a field and to perform appropriate irrigation control.

The present technology has been made in view of such a situation, and an object thereof is to acquire information about a moisture amount in a field and grasp a change in moisture amount over time, to make it possible to perform appropriate irrigation.

### Solutions to Problems

A first data processing apparatus according to one aspect of the present technology is a data processing apparatus including: a first creation unit configured to create a relative moisture amount map regarding a relative moisture amount in a field; an acquisition unit configured to acquire moisture amount data from a moisture amount sensor installed in the field; and a second creation unit configured to convert the relative moisture amount into an absolute moisture amount by using the moisture amount data, to create an absolute moisture amount map.

A data processing method according to one aspect of the present technology is a data processing method in which a data processing apparatus creates a relative moisture amount map regarding a relative moisture amount in a field, acquires moisture amount data from a moisture amount sensor installed in the field, and converts the relative moisture amount into an absolute moisture amount by using the moisture amount data, to create an absolute moisture amount map.

A second data processing apparatus according to one aspect of the present technology is a data processing apparatus for creating a soil moisture amount map indicating a distribution of a soil moisture amount in a field, on the basis of data related to soil moisture acquired for a first region of the field and data of a plurality of soil moisture sensors acquired for a plurality of regions that is included in the first region and is narrower than the first region.

A third data processing apparatus according to one aspect of the present technology is a data processing apparatus for generating a first soil moisture amount map on the basis of data related to soil moisture and acquired for a first region of a field, in which the first soil moisture amount map is generated to include a plurality of areas having relatively different soil moisture amounts.

A fourth data processing apparatus according to one aspect of the present technology is a data processing apparatus for calculating a soil moisture amount at a first point of a field, on the basis of measurement data of a soil electrical conductivity sensor acquired for the first point and measurement data of a soil moisture sensor acquired for a second point different from the first point.

In the first data processing apparatus and the data processing method according to one aspect of the present technology, a relative moisture amount map is created regarding a relative moisture amount in a field, moisture amount data is acquired from a moisture amount sensor installed in the field, and the relative moisture amount is converted into an absolute moisture amount by using the moisture amount data to create an absolute moisture amount map.

In the second data processing apparatus according to one aspect of the present technology, a soil moisture amount map is created indicating a distribution of a soil moisture amount in a field, on the basis of data related to soil moisture acquired for a first region of the field and data of a plurality of soil moisture sensors acquired for a plurality of regions that is included in the first region and is narrower than the first region.

The third data processing apparatus according to one aspect of the present technology is a data processing apparatus for generating a first soil moisture amount map on the basis of data related to soil moisture and acquired for a first region of a field, in which the first soil moisture amount map is generated to include a plurality of areas having relatively different soil moisture amounts.

In the fourth data processing apparatus according to one aspect of the present technology, a soil moisture amount at a first point of a field is calculated on the basis of measurement data of a soil electrical conductivity sensor acquired for a first point and measurement data of a soil moisture sensor acquired for a second point different from the first point.

Note that the data processing apparatus may be an independent apparatus or an internal block included in one apparatus.

### BRIEF DESCRIPTION OF DRAWINGS

FIG. 1 is a diagram illustrating a configuration example of an embodiment of a data processing system to which the present technology is applied.

FIG. 2 is a diagram illustrating an internal configuration example of the data processing system.

FIG. 3 is a diagram illustrating an internal configuration example of the data processing system.

FIG. 4 is a diagram illustrating a configuration example of a moisture sensor.

FIG. 5 is a diagram illustrating a configuration example of a measurement unit.

FIG. 6 is a flowchart for explaining processing of the data processing system.

FIG. 7 is a flowchart for explaining processing related to creation of a relative soil moisture amount map.

FIG. 8 is a flowchart for explaining processing related to creation of an absolute soil moisture amount map.

FIG. 9 is a flowchart for explaining processing related to estimation of a relative soil moisture amount.

FIG. 10 is a flowchart for explaining processing based on a soil moisture amount.

FIG. 11 is a diagram illustrating an example of a field.

FIG. 12 is a diagram for explaining a collection location of a soil sample.

FIG. 13 is a diagram for explaining measurement of a soil electrical conductivity.

FIG. 14 is a diagram for explaining grouped regions.

FIG. 15 is a diagram for explaining an installation location of the moisture sensor.

FIG. 16 is a diagram for explaining processing related to estimation of a moisture amount.

FIG. 17 is a diagram illustrating an example of a distribution of a moisture amount after estimation.

FIG. 18 is a diagram for explaining an irrigation amount.

FIG. 19 is a diagram for explaining an irrigation system.

FIG. 20 is a diagram illustrating an example of a way to issue an alert.

FIG. 21 is a diagram for explaining processing in a case where there are variations in a moisture amount.

FIG. 22 is a diagram for explaining another method of generating the relative soil moisture amount map.

FIG. 23 is a diagram for explaining grouped regions.

FIG. 24 is a diagram for explaining an installation location of the moisture sensor.

FIG. 25 is a diagram for explaining measurement of a soil electrical conductivity.

FIG. 26 is a diagram for explaining an affected region.

FIG. 27 is a diagram for explaining an estimation region.

FIG. 28 is a diagram for explaining a conversion formula.

FIG. 29 is a diagram for explaining conversion from an electrical conductivity to a moisture amount by using the conversion formula.

FIG. 30 is a diagram for explaining a distribution of a moisture amount after conversion.

FIG. 31 is a diagram for explaining processing related to estimation of a moisture amount.

FIG. 32 is a diagram for explaining processing related to update of the relative soil moisture amount map.

FIG. 33 is a diagram for explaining a configuration example of a PC.

### MODE FOR CARRYING OUT THE INVENTION

Hereinafter, a mode for carrying out the present technology (hereinafter referred to as an embodiment) will be described.

### <Configuration of Irrigation Control System>

The present technology can be applied to, for example, a system that performs irrigation suitable for growing crops in a field. In the following description, as an example of a data processing system to which the present technology is applied, an irrigation control system for controlling irrigation in a field will be described as an example.

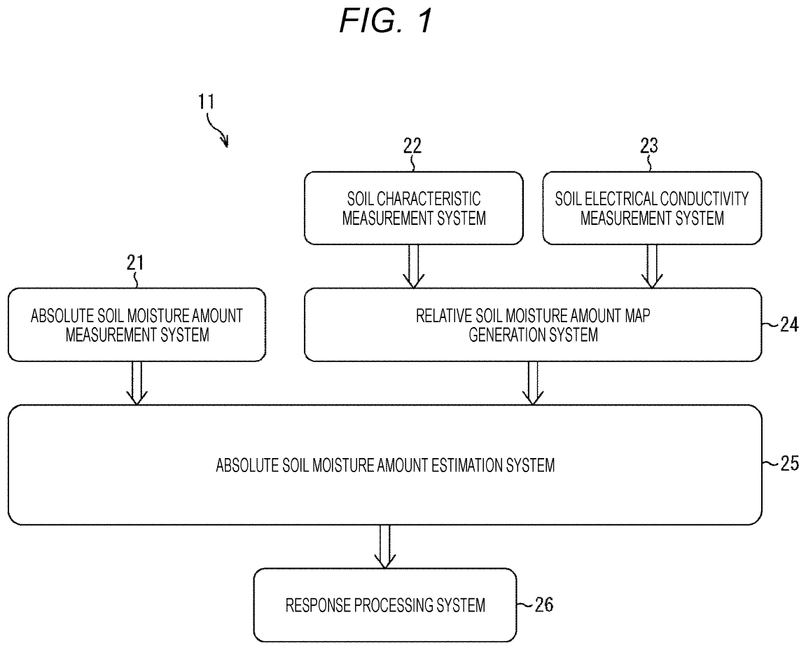

FIG. 1 is a diagram illustrating a configuration example of an irrigation control system 11 to which the present technology is applied. The irrigation control system 11 illustrated in FIG. 1 includes an absolute soil moisture amount measurement system 21, a soil characteristic measurement system 22, a soil electrical conductivity measurement system 23, a relative soil moisture amount map generation system 24, an absolute soil moisture amount estimation system 25, and a response processing system 26.

The absolute soil moisture amount measurement system 21 is a system including a moisture amount sensor installed in a field, and measures a value of a moisture amount at each point in the field. A sample may be collected and analyzed by an external system (analysis company). Furthermore, the absolute soil moisture amount measurement system 21 may be a system using both a system for measuring a moisture amount with a moisture sensor and a system for measuring a moisture amount for collecting a sample. In the following description, a case of a system including a moisture amount sensor will be described as an example.

The soil characteristic measurement system 22 measures characteristics of soil of the field, for example, a ratio between organic carbon, gravel, and silt, and viscosity in soil.

The soil electrical conductivity measurement system 23 measures an electrical conductivity of soil of the field.

The relative soil moisture amount map generation system 24 generates a map regarding a relative moisture amount of the field, by using data related to a moisture amount of soil and data related to characteristics of soil measured by the soil characteristic measurement system 22, and data related to an electrical conductivity measured by the soil electrical conductivity measurement system 23. As will be described in detail later, the map regarding a relative moisture amount of the field is a map in which the field is classified into a region having a large moisture amount in the field and a region having a small moisture amount with respect to the region having a large moisture amount.

The absolute soil moisture amount estimation system 25 estimates a moisture amount in the field by using data regarding an absolute moisture amount from the absolute soil moisture amount measurement system 21 and a relative soil moisture amount map generated by the relative soil moisture amount map generation system 24.

The response processing system 26 uses the moisture amount estimated by the absolute soil moisture amount estimation system 25 to determine whether or not it is necessary to take a response such as performing irrigation, and executes processing related to the response if necessary. For example, in a case where there is a region having a small moisture amount, irrigation is controlled so that the region has an appropriate moisture amount, or an alert for calling attention to the user is issued.

For example, by installing a moisture amount sensor that measures a moisture amount at predetermined intervals in the field, it is possible to measure a moisture amount in a predetermined region in the field. However, in a case where the field is large, it is difficult to install necessary and sufficient moisture amount sensors in the field because of efforts and costs.

The irrigation control system 11 to which the present technology is applied is a system that can accurately estimate (measure) a moisture amount of the entire field and perform irrigation according to the measured moisture amount, even in a case where a small number of moisture amount sensors are installed in the field. Furthermore, as will be described later, the system is also capable of performing estimation of a growth state of crops estimated from a moisture amount and processing according to a result of the estimation.

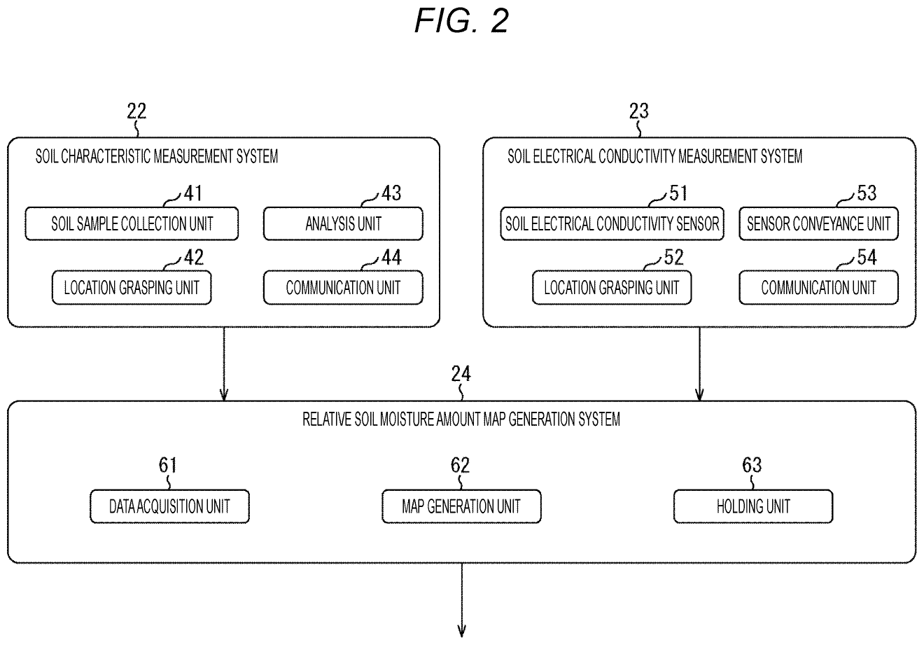

FIG. 2 is a diagram illustrating functional configuration examples of the soil characteristic measurement system 22, the soil electrical conductivity measurement system 23, and the relative soil moisture amount map generation system 24.

The soil characteristic measurement system 22 includes a soil sample collection unit 41, a location grasping unit 42, an analysis unit 43, and a communication unit 44. The soil sample collection unit 41 acquires soil in the field as a sample. The soil sample collection unit 41 includes, for example, a soil sampling tube (soil sampler) that collects soil from a ground surface to a predetermined depth. The soil sample collection unit 41 acquires the soil sample from a plurality of locations in the field by using the soil sampling tube. The processing related to acquisition of the soil sample may be performed by a user using the soil sampling tube, or may be automatically performed by a predetermined device without bothering the user.

The location grasping unit 42 grasps a location where the soil has been collected by the soil sample collection unit 41. When soil is collected by the soil sample collection unit 41 using, for example, a global navigation satellite system (GNSS), the location grasping unit 42 acquires a location thereof, and manages the soil sample and the location information in association with each other.

The analysis unit 43 analyzes the soil sample collected by the soil sample collection unit 41, and measures a ratio between organic carbon, gravel, silt, and viscosity in the soil, a component (ion concentration) of fertilizer, and the like. This analysis may be performed by requesting a company that analyzes the soil samples.

For example, the communication unit 44 controls communication when the location grasping unit 42 performs communication required when using the GNSS or transmits a result analyzed by the analysis unit 43 to another system or server (not illustrated) included in the irrigation control system 11.

The soil electrical conductivity measurement system 23 includes a soil electrical conductivity sensor 51, a location grasping unit 52, a sensor conveyance unit 53, and a communication unit 54. As the soil electrical conductivity sensor 51, it is possible to use an electromagnetic induction (EMI) sensor having a transmission coil that generates a magnetic field and a reception coil that senses an external magnetic field. The soil electrical conductivity sensor 51 is a sensor that measures the electrical conductivity of soil by measuring a secondary magnetic field by, for example, electromagnetic induction. An underground Doppler radar may be used as the soil electrical conductivity sensor 51.

The location grasping unit 52 grasps a location where the electrical conductivity of soil is measured by the soil electrical conductivity sensor 51. For example, when the soil electrical conductivity sensor 51 measures an electrical conductivity of soil by using GNSS, the location grasping unit 52 acquires a location thereof, and manages the electrical conductivity and the location data in association with each other.

The sensor conveyance unit 53 conveys the soil electrical conductivity sensor 51. The soil electrical conductivity sensor 51 is held by the user, for example, and moves in the field in the held state to measure the electrical conductivity. Alternatively, the soil electrical conductivity sensor 51 measures an electrical conductivity by, for example, moving in the field while being towed by a tractor. In such a case, the sensor conveyance unit 53 includes a person (a device that includes the soil electrical conductivity sensor 51 and can be held by a person) and the tractor.

The sensor conveyance unit 53 may be a drone. The electrical conductivity of the field may be acquired by hanging the soil electrical conductivity sensor 51 from the drone and moving close to the ground.

For example, the communication unit 54 controls communication when the location grasping unit 52 performs communication required when using the GNSS or transmits a result measured by the soil electrical conductivity sensor 51 to another system or server (not illustrated) included in the irrigation control system 11.

The relative soil moisture amount map generation system 24 includes a data acquisition unit 61, a map generation unit 62, and a holding unit 63. The data acquisition unit 61 acquires data of soil characteristics measured by the soil characteristic measurement system 22 and data of the electrical conductivity of soil measured by the soil electrical conductivity measurement system 23.

The map generation unit 62 generates a map representing a distribution of moisture on the basis of the soil characteristic data and the electrical conductivity data acquired by the data acquisition unit 61. The map generated by the map generation unit 62 is a relative soil moisture amount map.

The holding unit 63 holds (stores) the generated relative soil moisture amount map, and holds (stores) the data acquired by the data acquisition unit 61 as necessary.

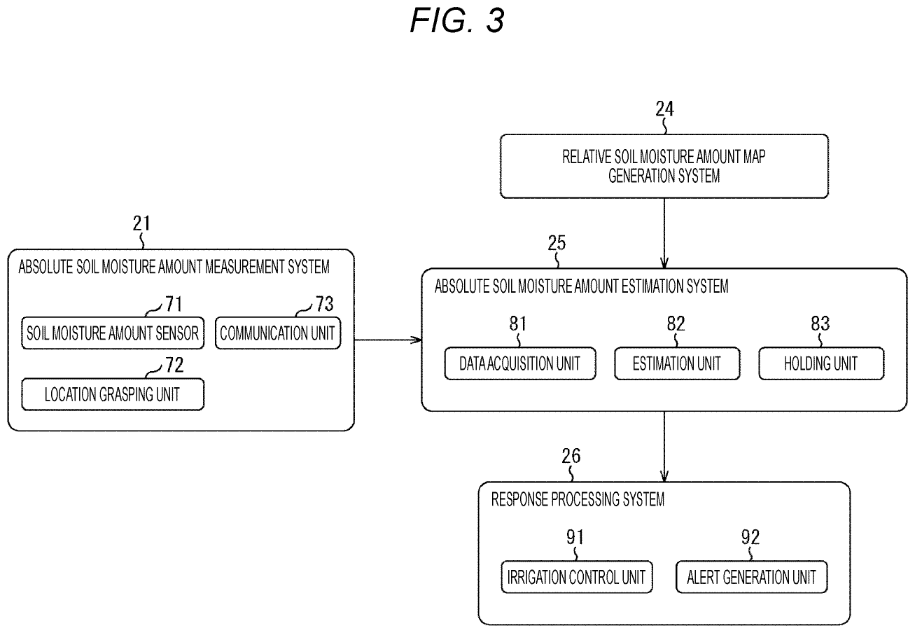

FIG. 3 is a diagram illustrating functional configuration examples of the absolute soil moisture amount measurement system 21, the absolute soil moisture amount estimation system 25, and the response processing system 26.

The absolute soil moisture amount measurement system 21 includes a soil moisture amount sensor 71, a location grasping unit 72, and a communication unit 73. The soil moisture amount sensor 71 is a sensor partially or entirely buried in soil and configured to measure a moisture amount of the soil. Here, an absolute moisture amount is a moisture amount at the time of measurement at the measurement point.

The location grasping unit 72 grasps a location where the soil moisture amount sensor 71 is installed. The location grasping unit 72 periodically grasps the location where the soil moisture amount sensor 71 is installed by using, for example, GNSS. When the soil moisture amount sensor 71 measures a moisture amount and transmits the measured moisture amount to another device, for example, a server (not illustrated), location information grasped by the location grasping unit 72 is transmitted in association with moisture amount data.

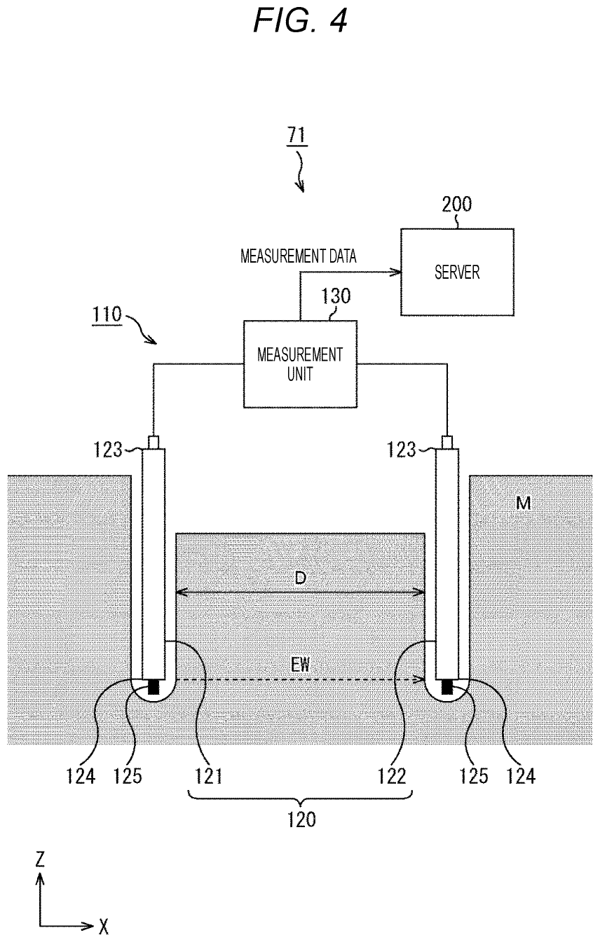

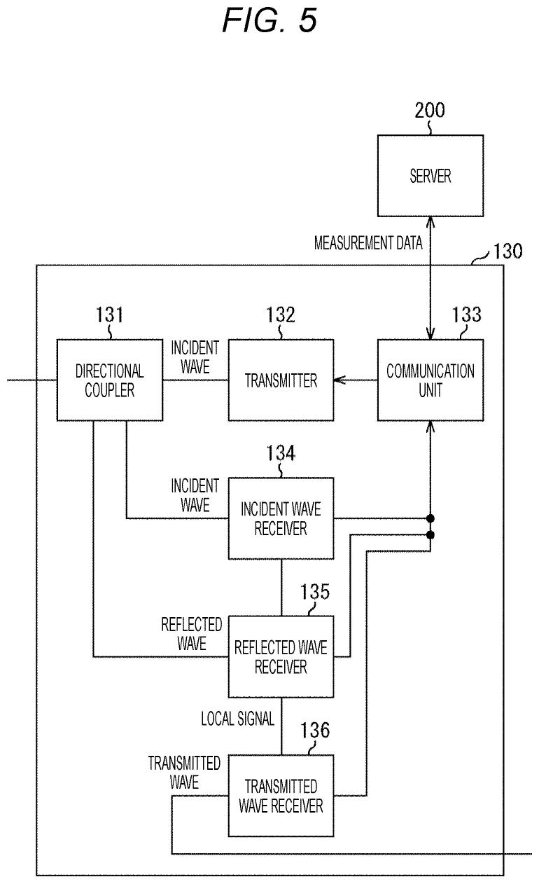

As the soil moisture amount sensor 71, a sensor having a configuration illustrated in FIGS. 4 and 5 can be used. The moisture amount sensor 71 measures a moisture amount contained in a medium M, and includes a sensor device 110. As the medium M, soil for growing crops is assumed, for example. The sensor device 110 acquires data necessary for measuring the moisture amount, as measurement data. The sensor device 110 transmits the measurement data to, for example, a server 200.

The server 200 can communicate with the sensor device 110, and is, for example, a terminal device used by an end user (an agricultural worker, an agricultural work manager, or the like), for example, a smartphone, a tablet computer, a personal computer, or the like. In this case, a signal from the sensor device 110 to the server 200 is transmitted by wireless communication. Alternatively, the server 200 may be built in a main body of the sensor device 110. In this case, a signal 409 from the sensor device 110 to the server 200 may be transmitted by wired communication or wireless communication. The server 200 measures the moisture amount by using the measurement data.

The sensor device 110 includes a sensor head 120 and a measurement unit 130. The sensor head 120 is a component including a pair of probes 121 and 122. These probes 121 and 122 are individually connected to the measurement unit 130 via cables and/or wires on a substrate. For example, a coaxial cable is used as the cables and/or the wires on the substrate. These cables and/or wires on the substrate are connected to the probes 121 and 122 by embedding respective tip ends inside the probes 121 and 122. The measurement unit 130 causes one of the probes 121 and 122 to transmit an electromagnetic wave EW and causes another one of the probes 121 and 122 to receive the electromagnetic wave EW to generate measurement data.

The sensor head 120 includes the probe 121 and the probe 122. A length of each of the probe 121 and the probe 122 is, for example, 75 to 150 millimeters (mm). A thickness (a diameter or a width of a probe cross section) of each of the probe 121 and the probe 122 is, for example, 3 to 30 millimeters (mm).

The probe 121 and the probe 122 are disposed in a medium such as soil, and each have an antenna 125 capable of transmitting and receiving an electromagnetic wave of a predetermined frequency between the probe 121 and the probe 122. The pair of probes 121 and 122 have an elongated rod shape, and each have a first end portion 123 and a second end portion 124. One of the first end portions 123 of the probe 121 and the probe 122 is connected with a transmitter 132 (FIG. 5) of the measurement unit 130, and another one of the first end portions 123 is connected with a transmitted wave receiver 136 (FIG. 5).

The second end portions 124 of the probe 121 and the probe 122 are spaced apart from the first end portions 123 in an axial direction Z of the probe 121 and the probe 122, and are insertion tip ends for the medium M. That is, the axial direction Z of the probe 121 and the probe 122 is an insertion direction into the medium M. The axial direction Z of the probe 121 and the probe 122 is a direction orthogonal to a separation direction X of the pair of antennas 125. The antenna 125 is provided at a location separated from the first end portion 123 in the axial direction Z of the probe 121 and the probe 122.

Specifically, the antenna 125 is provided at the second end portions 124 of the probe 121 and the probe 122. In other words, the antenna 125 is not provided widely over the axial direction Z of the probe 121 and the probe 122, but is provided at pinpoint in the axial direction Z. Therefore, as compared with a case where the antenna is provided widely over the axial direction Z of the probe, the antenna 125 has a narrow moisture amount measurement area in the axial direction Z and has a high spatial resolution in the axial direction Z.

The probe 121 and the probe 122 are buried in the medium in the axial direction Z such that a distance in the separation direction X between the respective antennas 125 is a predetermined value D. For example, the probe 121 and the probe 122 are embedded in the medium M in the axial direction Z in a substantially perpendicular posture. Note that, as long as the distance between the antennas 125 is D, positions of the probe 121 and the probe 122 are not limited to vertical positions.

In an orchard and the like, a case is conceivable in which a vertical hole is formed in the medium M by an excavator such as a shovel, and the probe 121 and the probe 122 are inserted into the medium M from an inner peripheral wall surface of the vertical hole. In this case, the axial direction Z (that is, an insertion depth direction) of the probe 121 and the probe 122 is not the vertical direction (longitudinal direction) but a horizontal direction (lateral direction). The present embodiment is also applicable to such a use case.

Magnitude of the distance D between the antennas 125 is not particularly limited. If the distance D is too long, attenuation of the electromagnetic wave EW propagating through the medium M increases, and there is a possibility that sufficient reception intensity cannot be obtained. Whereas, if the distance D is too short, observation may be technically difficult. In consideration of these, the distance D is set to an appropriate value. For example, the distance D is 25 to 75 millimeters (mm).

FIG. 5 illustrates a configuration example of the measurement unit 130. The measurement unit 130 has a directional coupler 131, the transmitter 132, a communication unit 133, an incident wave receiver 134, a reflected wave receiver 135, and the transmitted wave receiver 136. For example, a vector network analyzer is used as the measurement unit 130.

The directional coupler 131 separates electric signals that are transmitted through a cable and/or a wire on a substrate into an incident wave and a reflected wave. The incident wave is a wave of an electric signal transmitted by the transmitter 132, and the reflected wave is a wave obtained by reflecting the incident wave at a terminal end of the probe 121. The directional coupler 131 supplies the incident wave to the incident wave receiver 134 and supplies the reflected wave to the reflected wave receiver 135.

The transmitter 132 transmits an electric signal of a predetermined frequency as an electric signal to the probe 121 via the directional coupler 131 and the cable and/or the wire on the substrate. As the incident wave in the electric signal, for example, a continuous wave (CW) is used. The transmitter 132 sequentially switches a frequency by a step of 50 megahertz (MHz) within a frequency band of 1 to 9 gigahertz (GHz), for example, to transmit the electric signal.

The incident wave receiver 134 receives the incident wave from the directional coupler 131. The reflected wave receiver 135 receives the reflected wave from the directional coupler 131. The transmitted wave receiver 136 receives the transmitted wave from a probe 202. Here, the transmitted wave is obtained by the probe 202 converting, into an electric signal, an electromagnetic wave transmitted through the medium between the probe 121 and the probe 122.

The incident wave receiver 134, the reflected wave receiver 135, and the transmitted wave receiver 136 perform orthogonal wave detection and analog to digital (AD) conversion on the received incident wave, reflected wave, and transmitted wave, and transmit the result as measurement data to the server 200 via the communication unit 133.

The communication unit 133 in the measurement unit 130 illustrated in FIG. 5 can be used as the communication unit 73 of the absolute soil moisture amount measurement system 21 illustrated in FIG. 3. The location grasping unit 72 (FIG. 3) may be provided in the measurement unit 130. In a case where the communication unit 73 and the location grasping unit 72 are provided in the measurement unit 130, the measurement unit 130 is disposed on the ground instead of underground as illustrated in FIG. 4. The communication unit 73 and the location grasping unit 72 may be disposed on the ground and connected by a cable to the measurement unit 130 disposed underground.

The description returns to the functional configuration example of the system illustrated in FIG. 3. The absolute soil moisture amount estimation system 25 includes a data acquisition unit 81, an estimation unit 82, and a holding unit 83. The data acquisition unit 81 acquires data of an absolute moisture amount measured by the absolute soil moisture amount measurement system 21 and data of a relative soil moisture amount map generated by the relative soil moisture amount map generation system 24.

The estimation unit 82 reflects the absolute moisture amount data on the relative soil moisture amount map to generate an absolute soil moisture amount map. Although details will be described later, a moisture amount in a predetermined region in the relative soil moisture amount map is estimated on the basis of the absolute moisture amount data, and a map regarding a moisture amount in the entire field is generated.

Both the relative soil moisture amount map and the absolute soil moisture amount map are maps related to a distribution of a moisture amount of soil. However, the relative soil moisture amount map is a map focusing on a distribution of the moisture amount of the soil, and the absolute soil moisture amount map is a map focusing on accuracy of the moisture amount of the soil.

The holding unit 83 holds (stores) the generated absolute soil moisture amount map, and holds (stores) the data acquired by the data acquisition unit 81 as necessary.

The response processing system 26 includes an irrigation control unit 91 and an alert generation unit 92. The irrigation control unit 91 controls irrigation so that irrigation according to a moisture amount is performed in the field, by using the absolute soil moisture amount map created by the absolute soil moisture amount estimation system 25.

For example, in a case where it is a timing to measure a soil conductivity, the alert generation unit 92 generates an alert notifying the user of the fact. In a case where it is detected that a problem has occurred in growth of crops, the alert generation unit 92 generates an alert notifying the user of the fact.

### <Operation of Irrigation Control System 11>

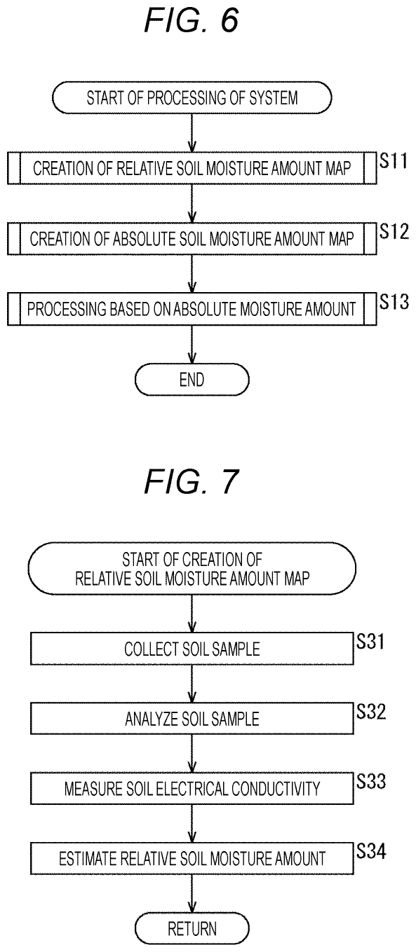

An operation of the irrigation control system 11 will be described with reference to flowcharts of FIGS. 6 to 10.

In step S11 (FIG. 6), a relative soil moisture amount map is created. The relative soil moisture amount map is performed by individual systems of the soil characteristic measurement system 22, the soil electrical conductivity measurement system 23, and the relative soil moisture amount map generation system 24 executing processing (described later) of a flowchart illustrated in FIG. 7.

The relative soil moisture amount map is a map indicating moisture in the field at a certain time. In other words, the relative soil moisture amount map is a map representing a distribution of a relative moisture amount when predetermined regions in the field are compared.

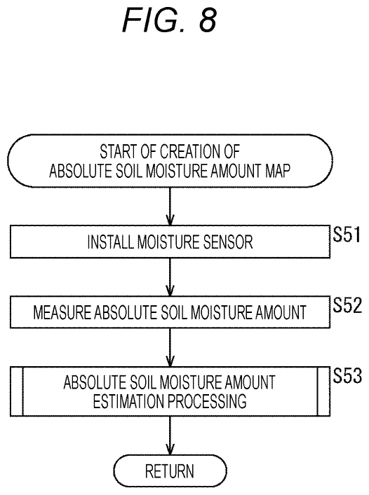

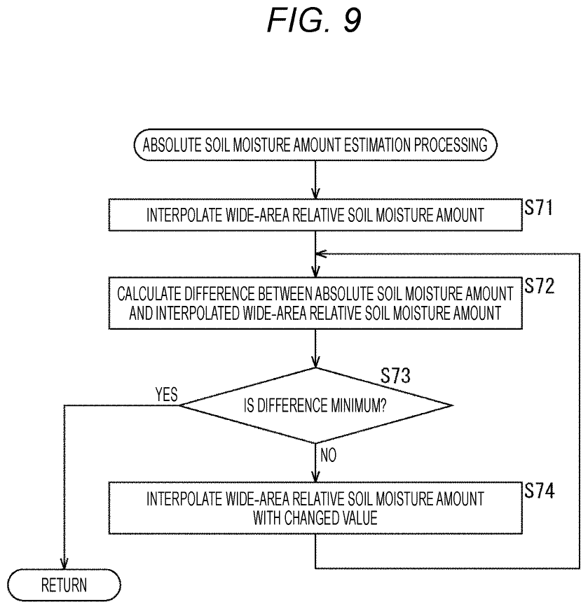

In step S12, an absolute soil moisture amount map is created. The absolute soil moisture amount map is performed by individual systems of the absolute soil moisture amount measurement system 21 and the relative soil moisture amount map generation system 24 executing processing (described later) of flowcharts illustrated in FIGS. 8 and 9.

The absolute soil moisture amount map is a map in which data related to a real-time moisture amount at the time of measurement in the field (moisture amount actually contained in soil at the time of measurement) is integrated. In other words, the absolute soil moisture amount map is a map representing a distribution of a moisture amount actually contained in a predetermined region in the field at that time when attention is paid to the region, and is a map in which a distribution of the moisture amount in a wide range is prioritized over accuracy of moisture amounts.

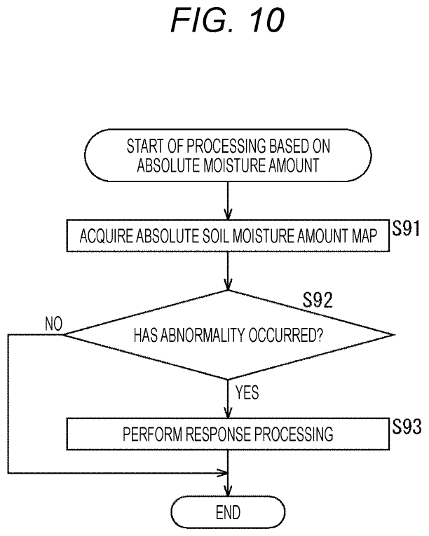

In step S13, processing based on an absolute moisture amount is executed by the response processing system 26. The processing based on an absolute moisture amount executed by the response processing system 26 will be described later with reference to a flowchart illustrated in FIG. 10.

### <Creation of Relative Soil Moisture Amount Map>

The generation of the relative soil moisture amount map executed in step S11 will be described with reference to the flowchart of FIG. 7.

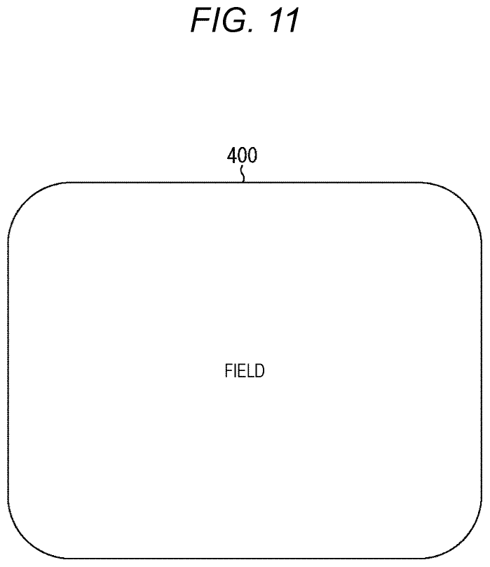

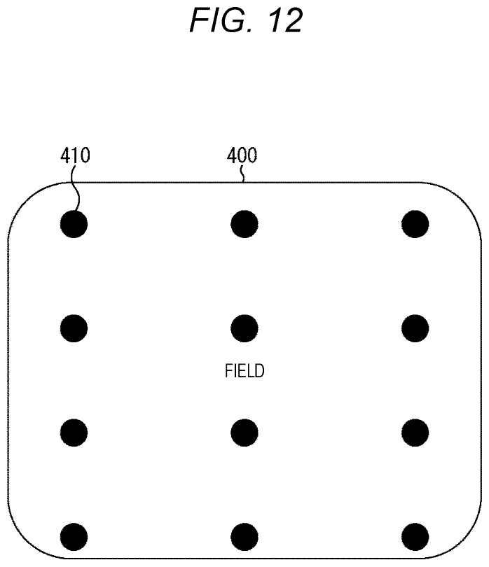

In step S31, the soil characteristic measurement system 22 acquires a soil sample. A case where a field 400 as illustrated in FIG. 11 is a target of the soil sample will be described as an example. As illustrated in FIG. 12, in the field 400, a soil sample is collected by using a device for collecting a soil sample such as a soil sampling tube at predetermined intervals.

The soil sample can be collected from a location where the soil sample does not affect growth of a tree body at a location avoiding crops (tree body). Therefore, the soil sample is not necessarily collected at places or locations at predetermined intervals, but may be collected randomly.

In the example illustrated in FIG. 12, a black spot represents a collection location 410 where the soil sample is collected. In the example illustrated in FIG. 12, there are 12 collection locations 410 in the field 400, and the soil sample is collected at the 12 collection locations 410. Note that one soil sample may be collected from the collection location 410, or a plurality of soil samples may be collected.

The collected soil sample and location information of the collection location 410 are managed in association with each other. When a plurality of soil samples is collected from the field 400, the processing proceeds to step S32 (FIG. 7), and the collected soil samples are analyzed.

By the analysis of the soil sample in step S32, a soil moisture amount, an ion concentration in the soil, for example, a concentration of nitrogen, phosphoric acid, and the like required for growth of crops, a PH value, and the like are acquired. Furthermore, a ratio of types of soil such as gravel, silt, and clay is also acquired. The analysis in step S32 may be performed by a company that performs analysis.

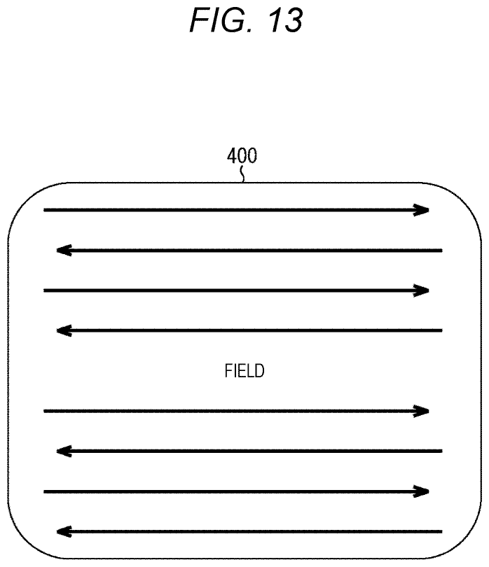

In step S33, the soil electrical conductivity measurement system 23 measures an electrical conductivity of the field 400. The electrical conductivity is measured, for example, by attaching the soil electrical conductivity sensor 51 to a tractor (the sensor conveyance unit 53) and causing the tractor to travel along a ridge in the field 400 as illustrated in FIG. 13. The electrical conductivity is measured in a state of being linked to location information acquired from a GPS or the like, by the tractor traveling and scanning in the field 400.

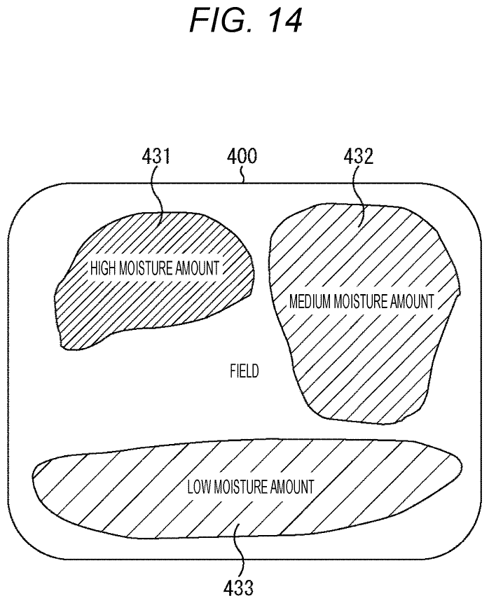

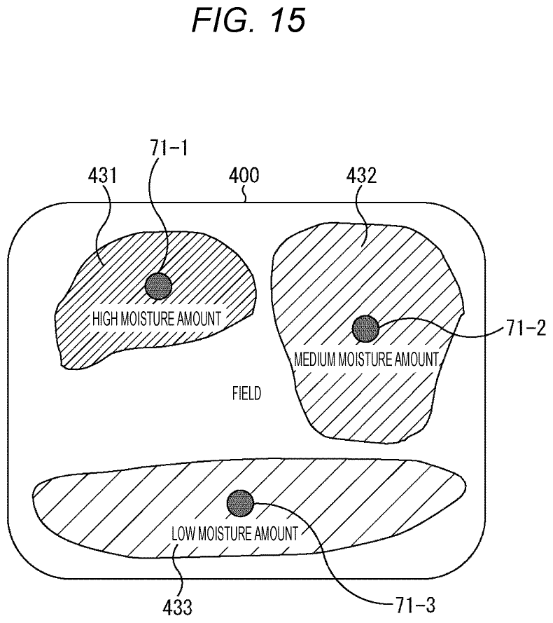

When the soil electrical conductivity is measured, the processing proceeds to step S34 (FIG. 7), and a relative soil moisture amount is estimated. As a result of estimating the relative soil moisture amount, a relative soil moisture amount map as illustrated in FIG. 14 is generated. In the example illustrated in FIG. 14, an example is illustrated in which regions related to three moisture amounts are generated in the field 400. A region 431 is a region estimated to have a high moisture amount, a region 432 is a region estimated to have a medium moisture amount, and a region 433 is a region estimated to have a small moisture amount.

The region 431 estimated to have a high moisture amount is a region estimated to have a larger moisture amount than the region 432 estimated to have a medium moisture amount or the region 431 estimated to have a small moisture amount. In other words, the region 431 is a region having a relatively larger moisture amount than the region 432 and the region 431, and is a region estimated to have a high water retention capacity. Similarly, the region 432 estimated to have a medium moisture amount is a region estimated to have a relatively larger moisture amount than the region 431 estimated to have a small moisture amount, and is a region estimated to have a large water retention capacity.

As described above, the relative soil moisture amount map is a map representing a relative value such as a large moisture amount or a small moisture amount in a case where moisture amounts in predetermined regions in the field 400 are compared.

The soil electrical conductivity is used as a value representing a fertilizer component and a salinity concentration contained in soil. It is known that the soil electrical conductivity is proportional to a moisture amount and an ion concentration. Therefore, if the ion concentration is specified, the moisture amount of the soil can be specified from the measured value of the electrical conductivity of the soil.

The ion concentration is acquired by collection and analysis of the soil sample. The soil electrical conductivity is measured as described above. It is known that a soil electrical conductivity and a soil moisture amount are in a proportional relationship, and a slope thereof depends on soil (ion concentration). By measuring an electrical conductivity with different soil moisture amounts, the slope of the relationship in the soil can be obtained. Thereafter, the moisture amount in the soil can be obtained by measuring the soil electrical conductivity in a wide range.

The collection and analysis of the soil sample executed in step S31 and step S32 are only required to be performed once at the beginning. The measurement of the soil electrical conductivity executed in step S33 is performed every time a predetermined period elapses. For example, the measurement is performed every three months. Furthermore, as described later, the measurement may be performed in accordance with a growth state of crops.

### <Creation of Absolute Soil Moisture Amount Map>

When the relative soil moisture amount map is created in step S11 (FIG. 6), the relative soil moisture amount map is used to create the absolute soil moisture amount map in step S12. The creation of the absolute soil moisture amount map executed in step S12 will be described with reference to the flowchart illustrated in FIG. 8.

In step S51, a moisture amount sensor is installed in the field 400. As the soil moisture amount sensor 71, the soil moisture amount sensor 71 described with reference to FIGS. 4 and 5 can be used. In the installation of the soil moisture amount sensor 71, a hole is made at a predetermined location in the field 400 by the user, and the soil moisture amount sensor 71 is embedded in the hole.

It takes efforts and costs to install a necessary and sufficient number of soil moisture amount sensors 71 in the field 400. By applying the present technology, by installing several soil moisture amount sensors 71 in the field 400, it is possible to obtain data equivalent to that in a case of installing a necessary and sufficient number of soil moisture amount sensors 71 in the field 400. Therefore, by applying the present technology, efforts and costs can be greatly reduced.

The soil moisture amount sensors 71 may be disposed at predetermined locations in the field 400, for example, at predetermined intervals, or a predetermined number of the soil moisture amount sensors 71 may be disposed in regions grouped into the regions 431 to 433 in the relative soil moisture amount map. For example, as illustrated in FIG. 15, one soil moisture amount sensor 71 can be installed in each of regions grouped into the regions 431 to 433.

In the example illustrated in FIG. 15, a soil moisture amount sensor 71-1 is installed in the region 431, a soil moisture amount sensor 71-2 is installed in the region 432, and a soil moisture amount sensor 71-3 is installed in the region 433. In a case where one soil moisture amount sensor 71 is installed in a region, the soil moisture amount sensor 71 may be installed near a barycenter of the region. In such a case, the barycenter is calculated for each region, and the soil moisture amount sensor 71 is installed at the calculated barycenter. The installation of the soil moisture amount sensor 71 does not necessarily coincide with the barycenter, and is only required to be in the vicinity of the barycenter. For example, it suffices that the soil moisture amount sensor 71 is installed in the vicinity of a barycentric position that does not interfere with growth of crops.

It suffices that the processing of installing the soil moisture amount sensor 71 in step S51 is executed only once. As described later, the soil moisture amount sensor 71 may be further added after the installation in some cases.

In step S52, the installed soil moisture amount sensor 71 measures a moisture amount of soil. The measurement of the moisture amount by the soil moisture amount sensor 71 is performed at predetermined intervals, for example, every 15 minutes, every one hour, or the like, and measured moisture amount data is supplied to the absolute soil moisture amount estimation system 25 (FIG. 3) via the communication unit 73 (FIG. 3).

In step S53, absolute soil moisture amount estimation processing is executed. The absolute soil moisture amount estimation processing is performed every time the soil moisture amount is measured. The absolute soil moisture amount estimation processing executed in step S53 will be described with reference to the flowchart of FIG. 9.

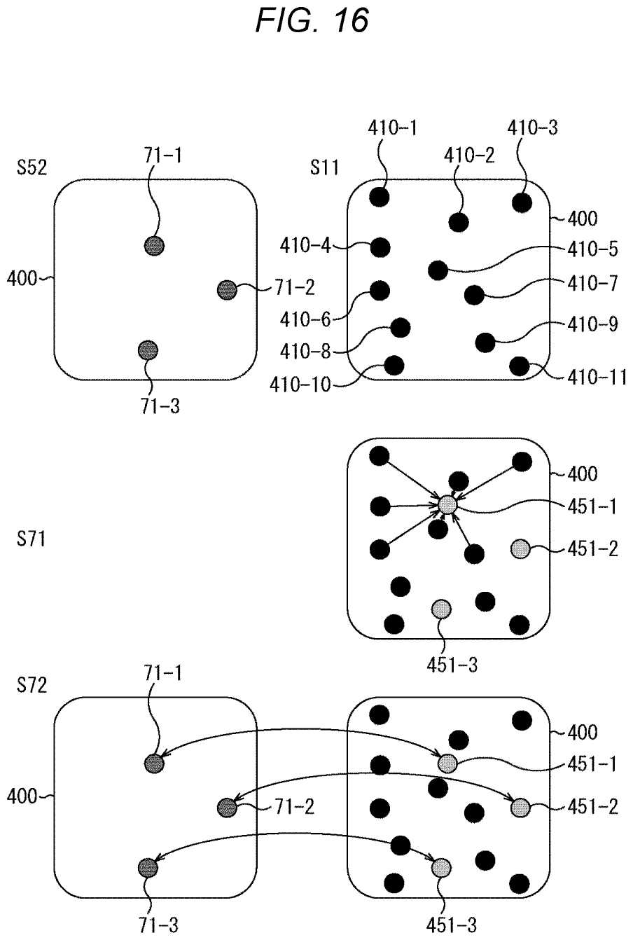

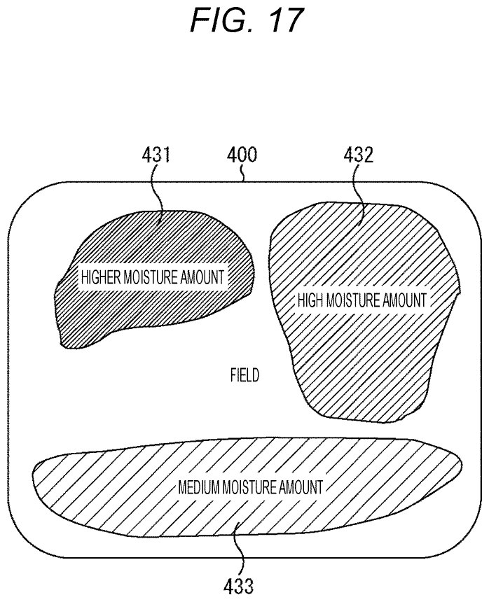

In step S71, a wide-area relative soil moisture amount is interpolated. The interpolation of the wide-area relative soil moisture amount will be described with reference to FIG. 16. The upper left diagram of FIG. 16 is a diagram illustrating an arrangement example, in the field 400, of the soil moisture amount sensor 71 that transmits the moisture amount data in step S52 (FIG. 8). The upper right diagram of FIG. 16 illustrates sets of an electrical conductivity and a location that are discretely obtained when a soil electrical conductivity is measured when the relative soil moisture amount map is generated in step S11 (FIG. 6). This is because the soil electrical conductivity is obtained in an analog form, and is temporally and spatially discrete by being digitized at the time of recording and acquired using GPS/GNSS.

Referring to the upper left diagram in FIG. 16, three sensors of the soil moisture amount sensor 71-1, the soil moisture amount sensor 71-2, and the soil moisture amount sensor 71-3 are installed in the field 400, and moisture amount data is acquired from each of the soil moisture amount sensors 71. Hereinafter, data of the soil moisture amount measured by the soil moisture amount sensor 71 will be described as absolute moisture amount data 71 as necessary.

The upper right diagram of FIG. 16 illustrates a spot where the electrical conductivity is discretely measured together with the location in the field 400. As a result, the relative moisture amount is calculated from sets 410-1 to 410-11 including the electrical conductivities and the collection locations. Hereinafter, data of a relative moisture amount calculated using a soil sample re-output from the collection location 410 will be described as relative moisture amount data 410 as necessary.

In a case where the processing of step S71 is performed in the state illustrated in the upper left diagram and the upper right diagram of FIG. 16, data of a wide-area relative soil moisture amount is generated and interpolated at a predetermined location as illustrated in the middle diagram of FIG. 16. The predetermined location is a location corresponding to the location where the soil moisture amount sensor 71 is installed. The wide-area relative soil moisture amount data is generated for the location.

For example, in the middle diagram of FIG. 16, wide-area relative soil moisture amount data 451-1 is generated for a location 451-1 corresponding to the location where the soil moisture amount sensor 71-1 is installed. The wide-area relative soil moisture amount data 451 is data generated using the relative moisture amount data 410, and is data treated as the generated relative moisture amount data 410 as a result of collection and analysis of a soil sample from a location 451 corresponding to the location where the soil moisture amount sensor 71 is installed.

One piece of the wide-area relative soil moisture amount data 451 is generated using all the relative moisture amount data 410 acquired from the field 400. Alternatively, one piece of the wide-area relative soil moisture amount data 451 is generated by using a predetermined number of pieces of the relative moisture amount data 410 located around the location 451 (located within a predetermined range) for which the wide-area relative soil moisture amount data 451 is to be generated. Alternatively, one piece of the wide-area relative soil moisture amount data 451 may be generated by using the relative moisture amount data 410 acquired from within a grouped region including a location for which the wide-area relative soil moisture amount data 451 is generated.

In the example illustrated in the middle part of FIG. 16, the wide-area relative soil moisture amount data 451 is generated using the relative moisture amount data 410-1 to 410-7 obtained from around the location for which the wide-area relative soil moisture amount data 451 is generated. The wide-area relative soil moisture amount data 451-1 may be generated using all the relative moisture amount data 410-1 to 410-11 in the field 400.

The wide-area relative soil moisture amount data 451 can be, for example, an average value of the relative moisture amount data 410. A value obtained by weighting a distance from the location 451 to the location for which the relative moisture amount data 410 is acquired may also be used for the calculation.

As illustrated in the lower right diagram of FIG. 16, wide-area relative soil moisture amount data 451-1 to 451-3 are generated for locations 451-1 to 451-3 corresponding to the locations where the soil moisture amount sensors 71-1 to 71-3 are installed, respectively. Such processing is performed as processing of interpolating the wide-area relative soil moisture amount in step S71 (FIG. 9).

In step S72, a difference between the absolute soil moisture amount and the interpolated wide-area relative soil moisture amount is calculated. Reference is made again to FIG. 16. Referring to the lower left diagram and the lower right diagram of FIG. 16, a difference (defined as a difference “a”) is calculated between absolute moisture amount data 71-1 measured by the soil moisture amount sensor 71-1 and the wide-area relative soil moisture amount data 451-1 generated for the location 451-1.

A difference (defined as a difference “b”) is calculated between absolute moisture amount data 71-2 measured by the soil moisture amount sensor 71-2 and the wide-area relative soil moisture amount data 451-2 generated for the location 451-2. A difference (defined as a difference “c”) is calculated between absolute moisture amount data 71-3 measured by the soil moisture amount sensor 71-3 and the wide-area relative soil moisture amount data 451-3 generated for the location 451-3.

When the difference is calculated in step S72 (FIG. 9), the processing proceeds to step S73, and it is determined whether or not the difference is the minimum. Here, a case is exemplified in which the difference “a”, the difference “b”, and the difference “c” are calculated. In such a case, it is determined whether or not the difference is the minimum for each of the difference “a”, the difference “b”, and the difference “c”. Alternatively, a value obtained by adding the differences “a”, “b”, and “c” and an average value of the differences “a”, “b”, and “c” are further calculated, and it is determined whether or not the calculated value is the minimum.

Here, it has been described that it is determined whether or not the calculated value is the minimum, but processing may be adopted in which it is determined whether or not the calculated value falls within a predetermined value (a value close to 0).

In a case where it is determined in step S73 that the difference is not the minimum, the processing proceeds to step S74. In step S74, the wide-area relative soil moisture amount data 451 is calculated again by changing the value of the relative moisture amount data 410.

The relative moisture amount data 410 is a value representing a relative moisture amount. The description will be continued by exemplifying a case where a difference value is calculated by subtracting the wide-area relative soil moisture amount data 451 from the absolute moisture amount data 71 in step S72.

In a case where the difference value is a positive number, the value of the relative moisture amount data 410 is changed to a larger value in order to approach the value of the absolute moisture amount data 71. In a case where the difference value is a negative value, the value of the relative moisture amount data 410 is changed to a smaller value in order to approach the value of the absolute moisture amount data 71.

The wide-area relative soil moisture amount data 451 is calculated again using the changed value of the relative moisture amount data 410. The recalculated wide-area relative soil moisture amount data 451 is used to perform the processing in and after step S72 again.

Whereas, in a case where it is determined in step S73 that the difference is the minimum, the absolute soil moisture amount estimation processing illustrated in FIG. 9 (processing of step S53 (FIG. 8)) is ended, and the processing proceeds to step S13.

By performing such processing, data can be acquired in which the absolute moisture amount data 71 is reflected on the relative moisture amount data 410. The relative moisture amount data 410 is data related to a moisture amount although accuracy in the field 400 is low. By reflecting, in such data, the absolute moisture amount data 71 which is data of the moisture value actually measured in the field 400, conversion into the absolute moisture amount data 71 with higher accuracy is performed.

By such processing, the absolute soil moisture amount map regarding a moisture amount in the field 400 at the time when the moisture amount is measured by the soil moisture amount sensor 71 is generated. FIG. 17 illustrates an example of the generated absolute soil moisture amount map. Description will be made while comparing the absolute soil moisture amount map illustrated in FIG. 17 with the relative soil moisture amount map illustrated in FIG. 14.

Paying attention to the region 431, the region has a high moisture amount in the relative soil moisture amount map illustrated in FIG. 14, but has been changed to a region having a higher moisture amount in the absolute soil moisture amount map illustrated in FIG. 17.

Paying attention to the region 432, the region has a medium moisture amount in the relative soil moisture amount map illustrated in FIG. 14, but has been changed to a region having a high moisture amount in the absolute soil moisture amount map illustrated in FIG. 17. Paying attention to the region 433, the region has a low moisture amount in the relative soil moisture amount map illustrated in FIG. 14, but has been changed to a region having a medium moisture amount in the absolute soil moisture amount map illustrated in FIG. 17. This is because, while the accuracy of the relative moisture amount map is low, accuracy has been improved by performing sensor fusion with the soil moisture amount sensor.

According to the present technology, the soil moisture amount can be accurately obtained in a wide range by sensor fusion of a low-accuracy sensor (for example, the soil electrical conductivity sensor 51) in a wide range and a high-accuracy sensor (for example, the soil moisture sensor 71) in a narrow range.

As described above, by reflecting the absolute moisture amount data 71 from the soil moisture amount sensor 71 on the relative soil moisture amount map, the absolute soil moisture amount map can be generated.

Even if the number of soil moisture amount sensors 71 installed in the field 400 is small, the moisture amount of the entire field 400 can be estimated with high accuracy. Therefore, efforts and costs required for measuring the moisture amount can be reduced. Furthermore, the absolute moisture amount data 71 from the soil moisture amount sensor 71 can be set to be automatically acquired without bothering the user, the moisture amount in the field 400 can be checked relatively frequently, and processing such as irrigation to be described later can also be finely controlled.

When the absolute soil moisture amount map is created in step S12 (FIG. 6), the processing proceeds to step S13. In step S13, processing based on an absolute moisture amount is executed. The processing based on an absolute moisture amount executed in step S13 will be described with reference to the flowchart of FIG. 10.

In step S91, the response processing system 26 acquires the absolute soil moisture amount map from the absolute soil moisture amount estimation system 25 (FIG. 3). In step S92, it is determined whether or not an abnormality has occurred. Examples of the abnormality include a small moisture amount in a predetermined region in the field 400, a possibility of occurrence of growth failure, and the like.

In a case where it is determined in step S92 that an abnormality has occurred, the processing proceeds to step S93, and response processing is executed.

Here, the response processing executed by the response processing system 26 will be described. For example, in a case where a scheduled irrigation time has come, a moisture amount in the field 400 has become equal to or less than a predetermined value, and the like, the response processing system 26 executes processing for controlling irrigation in the field 400.

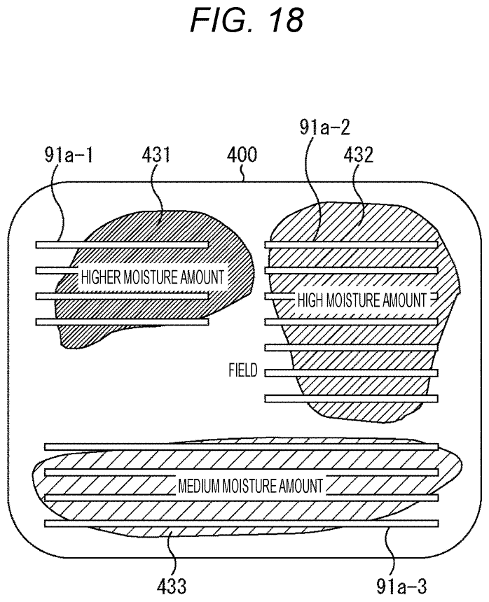

In a case where the absolute soil moisture amount map as illustrated in FIG. 17 is acquired and a scheduled irrigation time has come, control as illustrated in FIG. 18 is performed. In a case where it is desired to make the moisture amount in the field 400 uniform, the region 431 and the region 432 having a high moisture amount are irrigated with a small water amount, and the region 433 having a low moisture amount is irrigated with a large water amount.

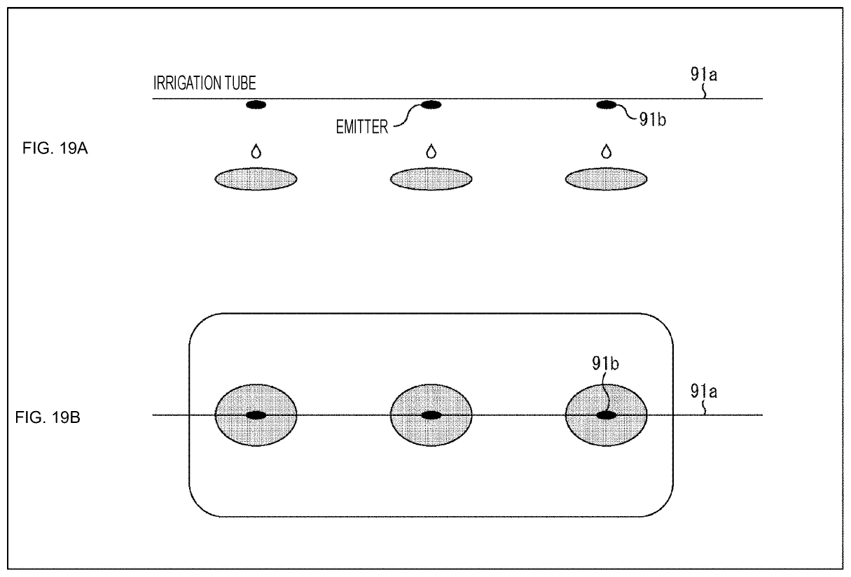

FIG. 19 is a diagram illustrating a configuration of a part of the irrigation control unit 91 that performs irrigation. A of FIG. 19 is a diagram when the irrigation control unit 91 is viewed from a lateral direction, and B of FIG. 19 is a diagram when the irrigation control unit 91 is viewed from an upper direction. The irrigation control unit 91 includes an irrigation tube 91a and an emitter 91b. The irrigation tube 91a is a tube through which water flows, and is provided with a plurality of the emitters 91b.

Water flowing in the irrigation tube 91a is poured from the emitter 91b into the field 400. A region where water drips from the emitter 91b is to have wet soil. As illustrated in B of FIG. 19, water spreads in a circular (elliptical) shape centered on the emitter 91b for irrigation.

Referring to FIG. 18, a plurality of the irrigation tubes 91a is disposed in the field 400. For example, the irrigation tube 91a is provided for each ridge and is disposed along the ridge. Control is performed to irrigate with a small water amount or not to irrigate, from an irrigation tube 91a-1 disposed in the region 431. Control is performed to irrigate with a small water amount from an irrigation tube 91a-2 disposed in the region 432. Control is performed to irrigate with a large water amount from an irrigation tube 91a-3 disposed in the region 433.

In this way, it is possible to perform appropriate irrigation according to the moisture amount actually contained. Here, the description has been given by taking irrigation as an example, but the present invention can also be applied to a case where a liquid fertilizer is supplied.

In a case where irrigation is performed using the irrigation tube 91a as illustrated in FIG. 19, soil immediately below the emitter 91b becomes wet soil. The irrigation tube 91a is disposed along a ridge and is not normally disposed between ridges. In such a case, soil that is wet by irrigation and soil that is not wet (dry) are mixed. The moisture amount of soil may be measured (estimated) in consideration of such a situation.

For example, the moisture amount of soil may be measured (estimated) in the assumption that there is moisture immediately below the emitter 91b and there is the same amount of moisture in soil immediately below another emitter 91b connected to the same irrigation tube 91a.

The soil moisture sensor 71 measures moisture immediately below the emitter 91b, and the soil moisture amount may be measured (estimated) in the assumption that the soil moisture amount immediately below the another emitter 91b connected to the irrigation tube 91a to which the emitter 91b is connected is the same at the same depth. Moreover, the moisture amount of soil may be measured (estimated) such that soil immediately below emitters 91b connected to the irrigation tubes 91a having the same irrigation amount is also treated to have the same moisture amount.

The processing may be performed assuming that there is no moisture between the emitters 91b from an implementation form of drip irrigation. In a case of drip irrigation, a location of a place of the emitter 91b can be specified by measuring with GPS or the like.

Irrigation causes soil immediately below the emitter 91b to contain moisture. The processing may be performed in the assumption that only moisture due to the irrigation is present in a period in which there is almost no rainfall and the like, and soil containing the moisture of the emitter 91b is discretely present around the emitter 91b as described with reference to FIG. 19. That is, the processing may be performed assuming that soil containing water exists discretely and there is a dry region having no water between the soil containing water.

In analyzing a distribution of the soil moisture amount, a wide-area relative soil moisture amount map is created using a soil sample for a map of a measured soil electrical conductivity (defined as a first condition). By applying installation information of the soil moisture sensor 71, a measurement result of the soil moisture sensor 71, and information about connection by the irrigation tube 91a for the emitter 91b immediately above the soil moisture sensor 71 with use of a map of the location of the emitter 91b, an absolute value (as well as an estimated value) of the soil moisture amount is measured (defined as a second condition).

A location of soil having a remarkably low moisture amount is estimated from location information between the emitters 91b. The moisture amount described above is measured by the soil moisture sensor 71 installed in a section other than the irrigation target in the field 400, so that a moisture amount of soil having a remarkably low moisture amount is estimated (defined as a third condition). By introducing the second condition and the third condition described above, the relative soil moisture amount map is corrected. By performing such correction, accuracy of the relative soil moisture amount map can be improved.

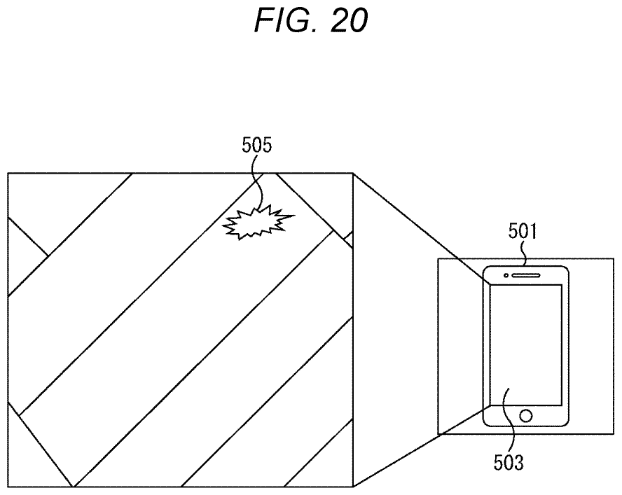

Another response processing executed in step S92 will be described with reference to FIG. 20. The response processing described with reference to FIG. 20 is processing of detecting that a problem has occurred in growth of crops and issuing an alert to an information terminal 501 of the user.

For example, an image obtained by aerially imaging the field 400 (a map including the field 400) is displayed on a display 503 of the information terminal 501 of the user. In a region where the problem has occurred in growth of crops, a mark 505 indicating the fact is displayed. When receiving such an alert, the user can actually move to the target individual, directly check the target individual, and take an appropriate action.

According to the present technology, it is possible to accurately estimate a moisture amount of the entire region in the field. Furthermore, it is possible to appropriately perform irrigation control and to manage a growth state of crops, by using the estimation result.

As a device for measuring an electrical conductivity of soil, for example, a device having two types of modes may be used. Mode 1 is an automated operation mode (a mode in which the device operates without bothering the user), which is, for example, a mode in which the device automatically goes around in the field 400 periodically while grasping a location of the device by the GNSS.

Mode 2 is a manual mode, which is a mode in which the device performs measurement by moving under human monitoring. Mode 2 also includes, for example, a case of a hand cart and a case of a drone operation with manual control.

For example, an alert may be issued, and the user who has received the alert may measure an electrical conductivity in the manual mode. Alternatively, an alert may be issued to a device that measures an electrical conductivity, and the electrical conductivity may be measured in the automatic mode.

A measuring device configured to measure an electrical conduction of soil and having the two modes holds a map of the field 400, information obtained by grouping the field 400 for each relationship between a soil electrical conductivity and a moisture amount, and a (plurality of) relationship between a soil electrical conductivity and a moisture amount, inside the measuring device or in an operation terminal that can communicate with the measuring device.

In a case where such a measuring device operates in the manual mode, when a boundary of a group is crossed, the user is notified through vibration of a handle or an operation terminal of the measuring device by a soil change notification function. The user can know that the relationship between the soil electrical conductivity and the moisture amount has changed together with tree vigor, without viewing the GUI.

Such a measuring device may be provided with a chapter function so that the following can be executed. When the user feels a change in tree vigor in a periphery of the measuring device, the user operates a predetermined button provided in the measuring device. By the button being operated, the measuring device can recognize a relationship between tree vigor and a soil moisture amount by displaying grouping information superimposed on the moisture amount map of the field during or after the measurement. A plurality of buttons or a function of switching roles of buttons in accordance with a reference of change (heavily diseased or wilted, or a type of bottom weed) may be provided.

A configuration may also be adopted in which a camera is mounted on the measuring device, and an image is recorded together with an estimated moisture amount in accordance with pressing of the button. At this time, the measured moisture amount may be stored in an Exif region of a captured image.

By using such a device, it is possible to intuitively know the relationship between tree vigor and soil.

### <Determination of Installation Location of Moisture Amount Sensor>

As a method of determining a location where the soil moisture amount sensor 71 is installed, as described with reference to FIG. 15, an example has been described in which the soil moisture amount sensor is installed at each barycentric position in the regions 431 to 433 grouped according to the moisture amount.

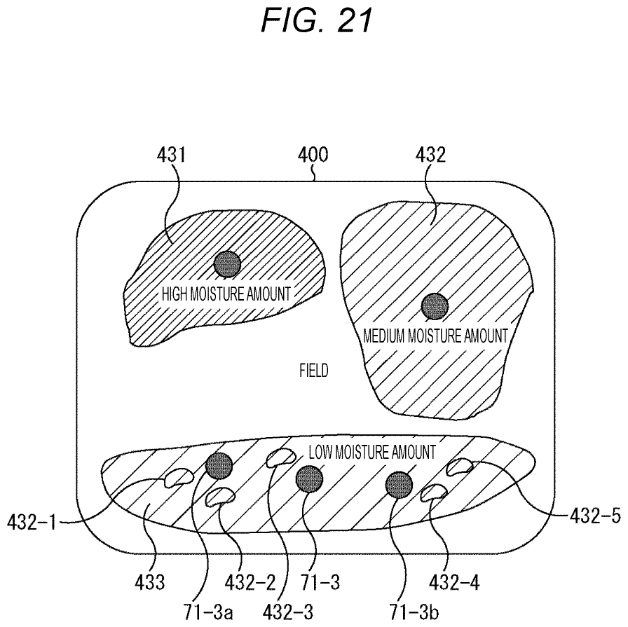

A case is also conceivable in which a variance of values in the grouped region is large, although grouping has been performed. For example, as illustrated in FIG. 21, in a case where regions 432-1 to 432-4 determined to have a medium moisture amount are scattered in the region 431 grouped as having a low moisture amount, the region 433 is a region having a large variance of values. In a such case where a variance of values in a region is large, the number of soil moisture amount sensors 71 installed in the region may be increased.

For example, in the example illustrated in FIG. 21, in the region 433, the soil moisture amount sensor 71-3 is installed at a barycentric position of the region 433, a soil moisture amount sensor 71-3a and a soil moisture amount sensor 71-3b are newly additionally installed, and a total of three soil moisture amount sensors 71 are installed.

As described above, after generation of the relative soil moisture amount map, in a case where there is a region having a variance of values in the region in the relative soil moisture amount map, a larger number of soil moisture amount sensors 71 may be installed in such a region than the number of soil moisture amount sensors 71 installed in a region in which there is no (a small) variance.

In a case where it is determined that there is a region having a large variance of values within the grouped region after the grouping, processing of finely grouping the region may be executed again. For example, in a case of a situation illustrated in an example illustrated in FIG. 21, the region 433 is determined to be a region having a large variance of values in the region, and processing for more finely grouping the region 433 is executed.

In this manner, grouping may be executed a plurality of times as necessary. In other words, there may be a flow of processing in which a coarse map is generated, and processing such as collection of a soil sample and measurement of a soil electrical conductivity is executed again for each region in the map, to create a detailed map.

### <Measurement of Soil Moisture Amount>

As a method of measuring the soil moisture amount, the following two methods can be considered. The first method is a method without calibration for soil moisture, and the second method is a method with calibration for soil moisture.

In the method without calibration for soil moisture, multiple investigations are conducted at different moisture levels in the same transect or site. The highest and lowest values of recorded soil electrical conductivity are used as bucket values “full” and “empty” for the site. Each site has its own “bucket size,” which can vary depending on the crop species. When a water fill rate is calculated at any given measurement time of any given site, a substantially linear response is obtained.

This approach is useful for explaining factors of unknown variations in testing, but requires periodic measurements throughout a planting period.

In the method with calibration for soil moisture, a soil electrical conductivity of wet soil is measured and a core is collected to determine an average VMC or moisture in units of mm. Similar processing is also performed in a dry state in a similar place.

VMC is a value calculated by the following formula.

$$VMC = ( water weight / water density ) / ( dry soil weight / bulk density )$$

As a result of this processing, a change in an actual soil electrical conductivity is obtained with respect to a penetration depth and a corresponding change in soil moisture.

This processing is performed in a plurality of places of the entire field, and whether or not an inclination has changed is determined. Further, one calibration is performed in the entire field or individual calibrations are performed in individual regions as necessary. This calibration can be executed retrospectively to determine actual soil moisture in a case where the technique “without calibration” has been previously used.

There is a linear correlation between a soil electrical conductivity and a soil moisture amount for every type of soil. As long as a graph is obtained for two points of a graph obtained by measuring in a dry state and a graph obtained by measuring in a wet state, a moisture amount in other cases can be estimated from a value of a soil electrical conductivity by using the graph.

In a case where the method with calibration for soil moisture is used out of the two methods listed as the method of measuring the soil moisture amount, core collection is performed. Since the core is collected to measure a moisture amount, the moisture amount can also be measured by the soil moisture amount sensor 71 instead of collecting the core. In this case, it is not necessary to collect the core for calibration.

A value of the installed soil moisture amount sensor 71 is monitored, and a soil electrical conductivity is measured when the moisture increases the most or when the moisture decreases the most. By determining a timing of measurement from a change in moisture, it is possible to measure, at a favorable timing, a soil electrical conductivity corresponding to each of a case of a high moisture amount and a case of a low moisture amount.

However, when the soil moisture amount sensor 71 containing metal is installed, there is a possibility that a measurement result of a soil electrical conductivity is affected in the vicinity of a spot where the soil moisture amount sensor is installed. Therefore, calibration in consideration of this possibility and measurement of the soil electrical conductivity avoiding the soil moisture amount sensor 71 are performed.

### <Another Method for Generating Relative Soil Moisture Amount Map>

Another method for generating the relative soil moisture amount map will be described. A case will be described in which the relative soil moisture amount map is generated using the method with calibration for soil moisture described above. In a case where the method with calibration for soil moisture is used, core collection is performed. However, a case will be described, as an example, in which a moisture amount is measured by the soil moisture amount sensor 71 instead of collecting the core.

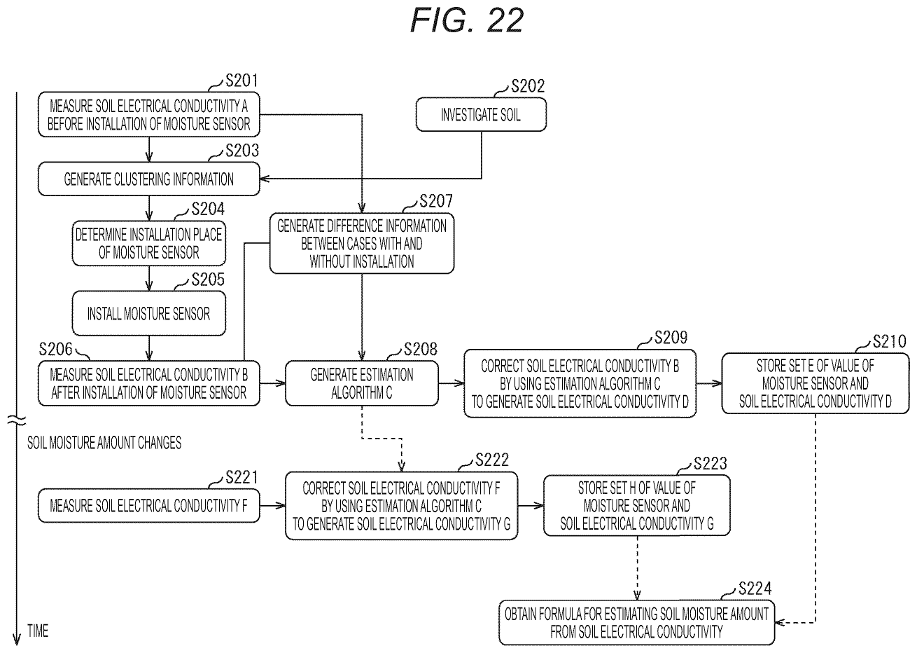

Another method for generating the relative soil moisture amount map will be described with reference to FIG. 22. In step S201, a soil electrical conductivity is measured. The processing in step S201 is executed in a state where the soil moisture amount sensor 71 is not installed. The soil electrical conductivity measured before installation of the soil moisture amount sensor 71 is described as Soil electrical conductivity A as needed.

The measurement of Soil electrical conductivity A is performed as described above with reference to step S33 (FIG. 7) and FIG. 13.

While Soil electrical conductivity A is measured, a soil investigation is performed in step S202. In the soil investigation, information is acquired such as a difference in soil, for example, good or poor drainage. By using a result of the soil investigation and Soil electrical conductivity A acquired in step S201, clustering information is generated in step S203.

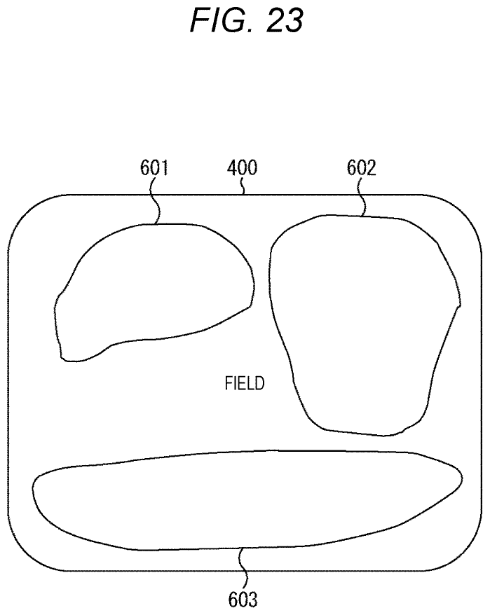

The clustering information is generated by clustering the result of the soil investigation and Soil electrical conductivity A into various distributions up to the number of regions corresponding to the number of soil moisture amount sensors 71 that can be installed in the field 400. FIG. 23 illustrates an example of the generated clustering information. The example illustrated in FIG. 23 illustrates a case where three distribution regions of a distribution region 601, a distribution region 602, and a distribution region 603 are generated in the field 400. Each distribution region is a result of integrating regions having similar soil characteristics, such as, for example, a region where drainage is good and a region where drainage is poor.

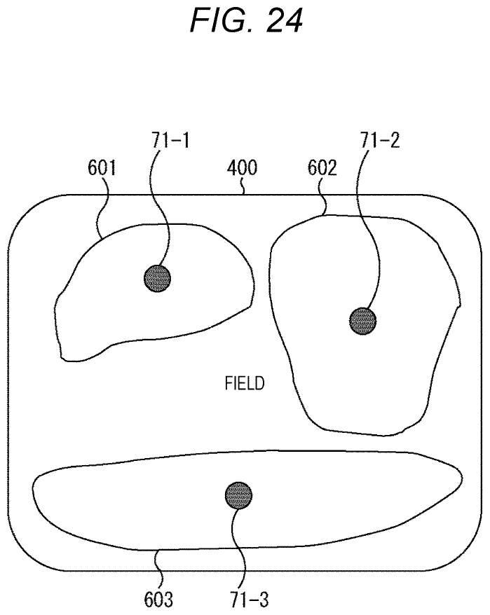

In step S204, an installation place of the soil moisture amount sensor 71 is determined. In step S205, the soil moisture sensor 71 is installed at the determined installation place. For example, the soil moisture amount sensor 71 is installed at a barycenter in a distribution region that is a result of clustering using cluster information. As illustrated in FIG. 24, the soil moisture amount sensor 71-1 is installed at a barycenter of the distribution region 601, the soil moisture amount sensor 71-2 is installed at a barycenter of the distribution region 602, and the soil moisture amount sensor 71-3 is installed at a barycenter of the distribution region 603.

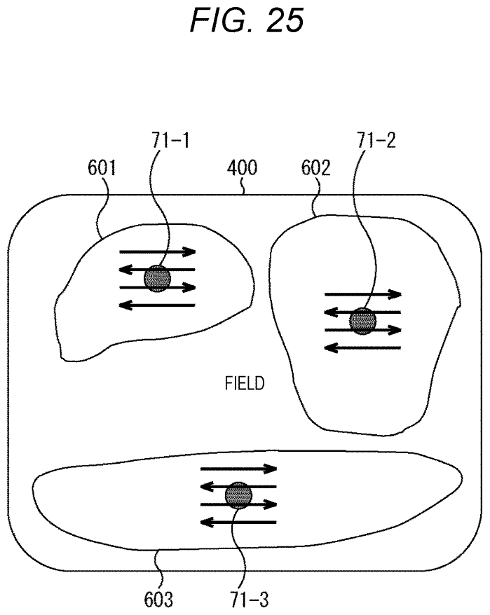

In step S206, after the installation of the soil moisture amount sensor 71, a soil electrical conductivity is measured. The soil electrical conductivity measured at a time point after the installation of the soil moisture amount sensor 71 is described as Soil electrical conductivity B as needed. The measurement of the soil electrical conductivity executed in step S206 is performed within a predetermined range centered on the location where the soil moisture amount sensor 71 is installed as illustrated in FIG. 25. Note that the soil electrical conductivity may be measured again for the entire field 400.

In step S207, information regarding an influence of the installation of the soil moisture amount sensor 71 is generated. Installation of the soil moisture amount sensor 71 may affect soil around the soil moisture amount sensor 71. Since the soil moisture amount sensor 71 has a structure including metal or plastic, there is a possibility that, for example, a soil electrical conductivity of soil around the location where the soil moisture amount sensor 71 is installed changes as compared with that before the installation.

In step S207, Soil electrical conductivity A before the installation of the soil moisture amount sensor 71 acquired in the processing of step S201 is compared with Soil electrical conductivity B after the installation of the soil moisture amount sensor 71 acquired in the processing of step S206, and information regarding the difference is generated. The difference information is used as information regarding a region affected by the soil moisture amount sensor 71.

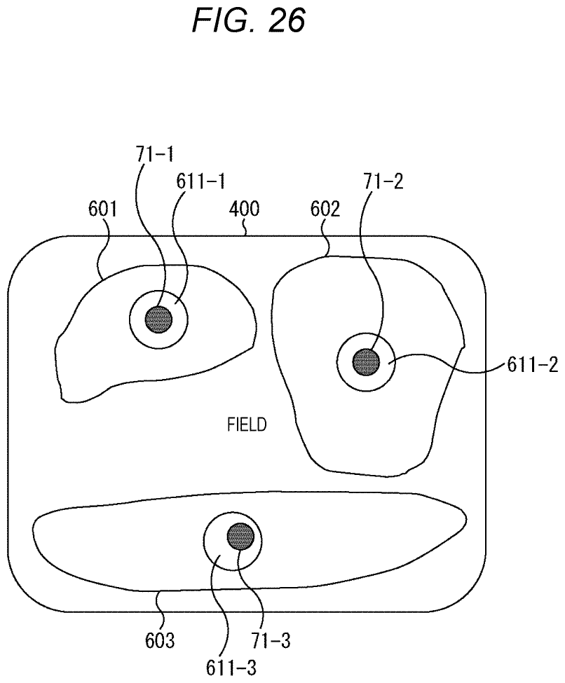

As illustrated in FIG. 26, for example, a predetermined range around the soil moisture amount sensor 71-1 is set as an affected region 611-1. Similarly, a predetermined range around the soil moisture amount sensor 71-2 is set as an affected region 611-2, and a predetermined range around the soil moisture amount sensor 71-3 is set as an affected region 611-3.

A center of the soil moisture amount sensor 71-1 and a center of the affected region 611-1 substantially coincide with each other. A center of the soil moisture amount sensor 71-2 and a center of the affected region 611-2 substantially coincide with each other. A center of the soil moisture amount sensor 71-3 and a center of the affected region 611-3 are shifted from each other.

An affected region 611 that may be affected by the installation of the soil moisture sensor 71 may be set as a region within a predetermined distance from the soil moisture sensor 71. By setting the affected region 611 by comparing the soil electrical conductivity before and after the installation of the soil moisture sensor 71 as described above, it is possible to specify the affected range more accurately than the case where the affected region 611 is simply set in accordance with the distance.

In step S208, Estimation algorithm C is generated using Soil electrical conductivity B acquired in the processing of step S206 and the difference information (information about the affected region 611) acquired in the processing of step S207. Estimation algorithm C is an algorithm for enabling acquisition of a soil electrical conductivity excluding the influence of the soil moisture amount sensor 71.

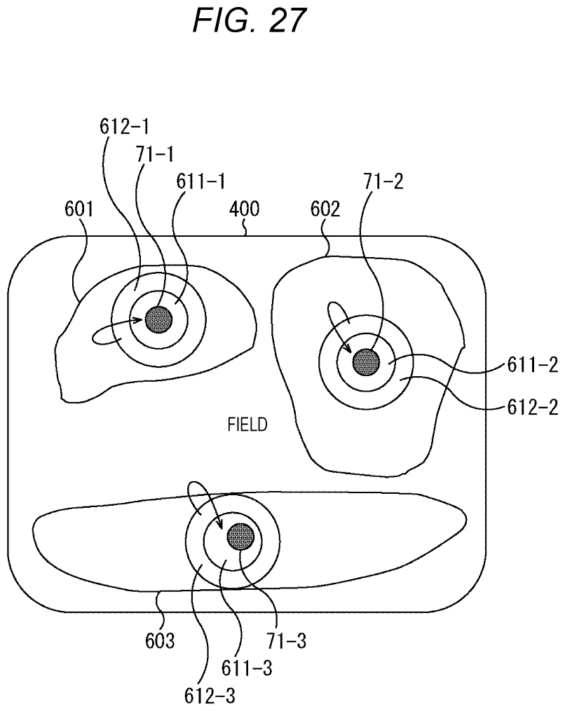

Generation of Estimation algorithm C will be described with reference to FIG. 27. For example, a region within a predetermined range around the affected region 611-1 is set as an estimation region 612-1 as a region in which a soil electrical conductivity in the affected region 611-1 is estimated. The soil electrical conductivity of the affected region 611-1 is estimated using a soil electrical conductivity acquired from within the estimation region 612-1. An algorithm used for this estimation, in other words, an algorithm for converting the soil electrical conductivity acquired from within the estimation region 612-1 into the soil electrical conductivity in the affected region 611-1 is generated as Estimation algorithm C.

Estimation algorithm C can be an algorithm for obtaining an average value of the soil electrical conductivity acquired from within the estimation region 612-1 or an algorithm obtained by regression analysis. An algorithm obtained by performing learning to correct Soil electrical conductivity A obtained before the installation of the soil moisture amount sensor 71 (step S201) by using a value of Soil electrical conductivity B obtained after installation of the soil moisture amount sensor 71 (step S206) may be used as Estimation algorithm C.

Referring to FIG. 27, Estimation algorithm C may be generated for each of the affected regions 611-1 to 611-3, or may be generated as an algorithm capable of supporting all the affected regions 611 in the field 400.

In the former case, in the situation as illustrated in FIG. 27, Estimation algorithm C for the affected region 611-1, Estimation algorithm C for the affected region 611-2, and Estimation algorithm C for the affected region 611-3 are individually generated. In the latter case, in the situation as illustrated in FIG. 27, Estimation algorithm C applicable to all the regions of the affected regions 611-1 to 611-3 is generated.

In step S209, Soil electrical conductivity B is corrected using Estimation algorithm C, to generate Soil electrical conductivity D. Soil electrical conductivity D in the affected region 611 is estimated from the acquired Soil electrical conductivity B on the basis of Estimation algorithm C.

Referring again to FIG. 27, Soil electrical conductivity B acquired from within the estimation region 612-1 and Estimation algorithm C are used to generate Soil electrical conductivity D in the affected region 611-1. Soil electrical conductivity B acquired from within of the estimation region 612-2 and Estimation algorithm C are used to generate Soil electrical conductivity D in the affected region 611-2. Soil electrical conductivity B acquired from within of the estimation region 612-3 and Estimation algorithm C are used to generate Soil electrical conductivity D in the affected region 611-3.

In step S210, absolute moisture amount data from the soil moisture amount sensor 71 and Soil electrical conductivity D (Soil electrical conductivity D in the affected region 611) estimated in step S209 are stored in association with each other. Set E in which the absolute moisture amount data and Soil electrical conductivity D are associated with each other is stored for each soil moisture amount sensor 71 installed in the field 400.

As described above, processing of steps S221 to S224 is executed at the time when it can be determined that the soil moisture amount has changed, at a time point after storing Soil electrical conductivity D in the affected region 611 around the soil moisture amount sensor 71. The time when it can be determined that the soil moisture amount has changed is, for example, when there is dry weather, rainfall, irrigation, or the like.

In step S221, a soil electrical conductivity is measured. In this measurement, similarly to the measurement of the soil electrical conductivity executed in step S206, as illustrated in FIG. 25, only a periphery of the location where the soil moisture amount sensor 71 is installed is measured. The soil electrical conductivity acquired by the processing in step S221 is described as Soil electrical conductivity F.

In step S222, the acquired Soil electrical conductivity F is corrected using Estimation algorithm C, whereby Soil electrical conductivity G of the affected region 611 around the soil moisture amount sensor 71 is acquired.

In step S223, the absolute moisture amount data from the soil moisture amount sensor 71 and Soil electrical conductivity G (Soil electrical conductivity G in the affected region 611) estimated in step S222 are stored in association with each other. Set H in which the absolute moisture amount data and Soil electrical conductivity D are associated with each other is stored for each soil moisture amount sensor 71 installed in the field 400.

Set E stored in the processing of step S210 and the Set H stored in the processing of step S223 are sets in which absolute moisture amount data acquired in different states and soil electrical conductivities are associated with each other. One of the different conditions is when soil is in a dry state and another one is when soil is in a wet state.

That is, by executing the processing of steps S201 to S210 and the processing of steps S221 to S223 in the different states, the relationship between the soil electrical conductivity and the soil moisture amount when soil is dry and the relationship between the soil electrical conductivity and the soil moisture amount when soil is wet are acquired.

As described above, there is a linear correlation between the soil electrical conductivity and the soil moisture amount for every type of soil. As long as a graph obtained by measurement in a dry state and a graph obtained by measurement in a wet state are obtained, a moisture amount in other cases can be estimated from a value of a soil electrical conductivity by using the graphs. Then, by executing the processing of the flowchart illustrated in FIG. 22, a graph obtained by performing measurement in a dry state and a graph obtained by performing measurement in a wet state are obtained.

In step S224, data of Set E stored in step S210 and Set H stored in step S223 are used to obtain a formula (graph) for estimating the soil moisture amount from the soil electrical conductivity. The graph is obtained for each soil moisture amount sensor 71 installed in the field 400.

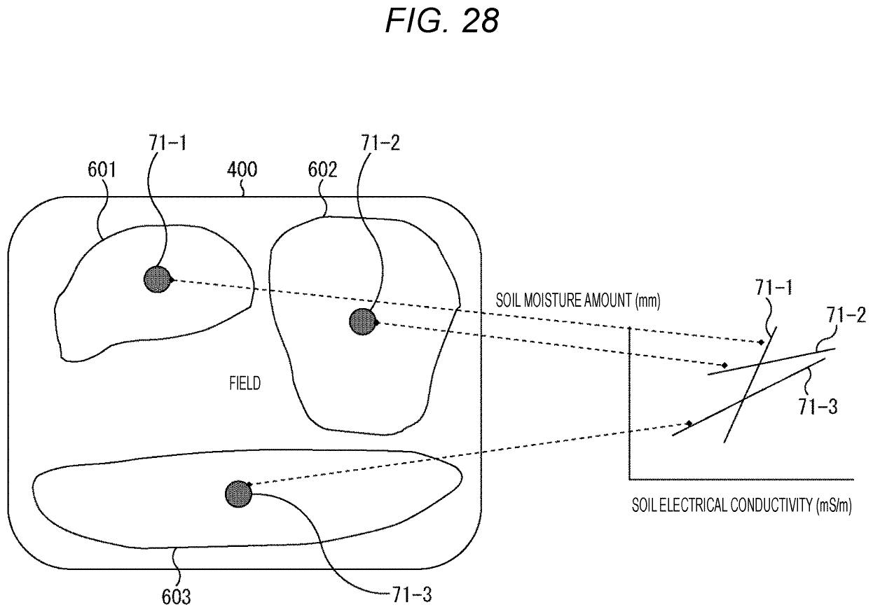

An example of the obtained graph is illustrated in FIG. 28. The right diagram of FIG. 28 is an example of the obtained graph. In the graph illustrated in FIG. 28, a vertical axis represents a soil moisture amount (mm), and a horizontal axis represents a soil electrical conductivity (mS/m). A graph 71-1 is obtained from the soil moisture amount sensor 71-1 installed in the field 400, a graph 71-2 is obtained from the soil moisture amount sensor 71-2, and a graph 71-3 is obtained from the soil moisture amount sensor 71-3.

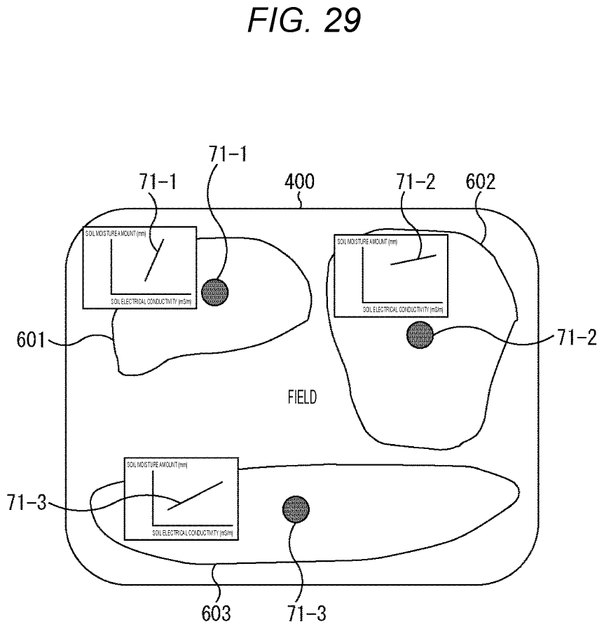

As described above, when the graph 71 is obtained for each soil moisture amount sensor 71, processing of converting the soil electrical conductivity into the soil moisture amount is executed using the graph. As illustrated in FIG. 29, the soil electrical conductivity measured in the distribution region 601 where the soil moisture amount sensor 71-1 is installed and the graph 71-1 (conversion formula 71-1) are used to calculate a moisture amount in the distribution region 601.

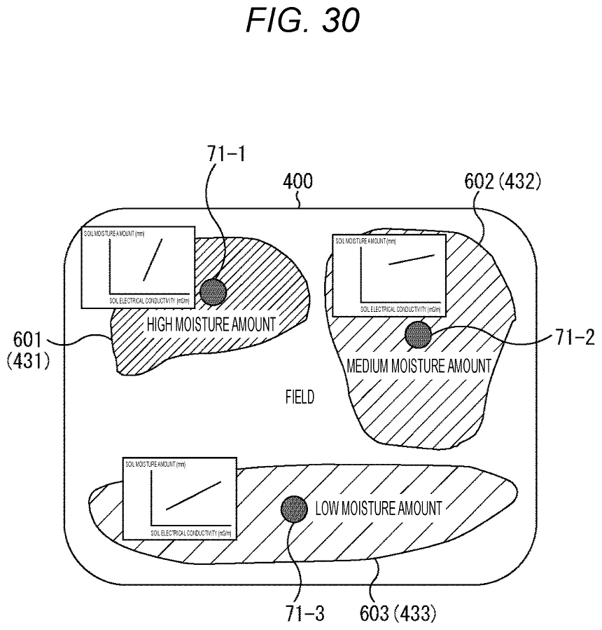

As a result of calculating the moisture amount in the distribution region 601, for example, as illustrated in FIG. 30, the distribution region 601 is set as a region having a high moisture amount. The distribution region 601 in which the moisture amount is set can be treated as a region corresponding to the region 431 of the relative soil moisture amount map described with reference to FIG. 15.

The soil electrical conductivity measured in the distribution region 602 where the soil moisture amount sensor 71-2 is installed and the graph 71-2 (conversion formula 71-2) are used to calculate a moisture amount in the distribution region 602. As a result of calculating the moisture amount in the distribution region 602, for example, as illustrated in FIG. 30, the distribution region 602 is set as a region having a medium moisture amount. The distribution region 602 in which the moisture amount is set can be treated as a region corresponding to the region 432 of the relative soil moisture amount map described with reference to FIG. 15.

The soil electrical conductivity measured in the distribution region 603 where the soil moisture amount sensor 71-3 is installed and the graph 71-3 (conversion formula 71-3) are used to calculate a moisture amount in the distribution region 603. As a result of calculating the moisture amount in the distribution region 603, for example, as illustrated in FIG. 30, the distribution region 603 is set as a region having a low moisture amount. The distribution region 603 in which the moisture amount is set can be treated as a region corresponding to the region 433 of the relative soil moisture amount map described with reference to FIG. 15.

In this way, by obtaining the moisture amount in each region of the distribution regions 601 to 603, it is possible to create the relative soil moisture amount map.

The processing of creating the relative soil moisture amount map corresponds to the processing of step S11 in the processing of the flowchart illustrated in FIG. 6. When the relative soil moisture amount map is generated in step S11, the processing proceeds to step S12. In step S12, an absolute soil moisture amount map is created. The processing of step S12 and subsequent steps is performed as described above with reference to FIG. 6 and subsequent figures.

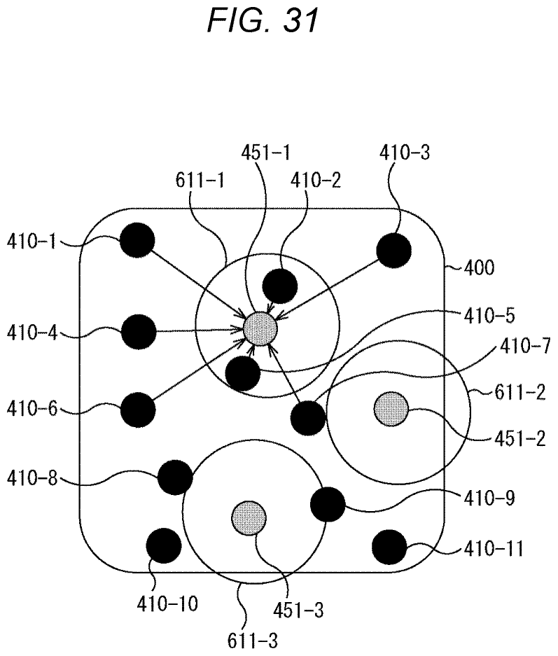

The generation of the absolute soil moisture amount map has been described with reference to the flowchart illustrated in FIG. 9 and FIG. 16. The processing of creating the absolute soil moisture amount map includes processing of interpolating the wide-area relative soil moisture amount data 451 (FIG. 16). The processing of interpolating the wide-area relative soil moisture amount data 451 will be described again with reference to FIG. 31.

FIG. 31 is a figure basically the same as the middle diagram of FIG. 16. In FIG. 31, the wide-area relative soil moisture amount data 451-1 is generated for a location 451-1 corresponding to the location where the soil moisture amount sensor 71-1 is installed.

Referring again to the example illustrated in the middle part of FIG. 16, the wide-area relative soil moisture amount data 451 is generated using the relative moisture amount data 410-1 to 410-7 obtained from around the location for which the wide-area relative soil moisture amount data 451 is generated. In this case, the relative moisture amount data 410 measured from the vicinity of the location where the soil moisture amount sensor 71-1 is installed is also used.

As described above, the soil electrical conductivity acquired from the region around the soil moisture amount sensor 71-1 may be affected by the soil moisture amount sensor 71-1. The relative moisture amount data 410 acquired from within the region that may be affected may be made not to be used at the time of generating the wide-area relative soil moisture amount data 451-1.

The region that may be affected by the soil moisture amount sensor 71-1 corresponds to the affected region 611-1 (FIG. 26). The relative moisture amount data 410 acquired from within the affected region 611-1 may be made not to be used at the time of generating the wide-area relative soil moisture amount data 451-1.

Referring to FIG. 31, there are relative moisture amount data 410-2 and relative moisture amount data 410-5 in the region set as the affected region 611-1 in the soil moisture amount sensor 71-1. In such a case, the wide-area relative soil moisture amount data 451-1 is generated using relative moisture amount data 410-1, 410-2 to 410-4, 410-6, and 410-7.

In this manner, the wide-area relative soil moisture amount data 451 may be generated by excluding the relative moisture amount data 410 in the affected region 611.

Alternatively, data estimated using Estimation algorithm C (FIG. 22) may be used as the relative moisture amount data 410 in the affected region 611.

In this way, the relative soil moisture amount map may be generated, and the absolute soil moisture amount map may be generated.

In a case of creating the relative soil moisture amount map for the first time, the relative soil moisture amount map is created as described with reference to FIG. 22. However, when the relative soil moisture amount map is updated thereafter, the relative soil moisture amount map is updated on the basis of processing described with reference to FIG. 31.

In step S301, a soil electrical conductivity is measured. This processing corresponds to step S206 in the processing illustrated in FIG. 22. In step S302, clustering information is acquired. The acquired clustering information is the clustering information generated in step S203 (FIG. 22). This clustering information is, for example, information representing the distribution regions 601 to 603 in the case described with reference to FIG. 23 and the like.

In step S305, the soil electrical conductivity for each cluster (each of the distribution regions 601 to 603) is acquired. In this processing, the soil electrical conductivity in the affected region 611 is interpolated by executing processing of correcting and acquiring the soil electrical conductivity in the affected region 611 by using Estimation algorithm C. This processing includes processing corresponding to the processing of step S209 or step S222 in the processing illustrated in FIG. 22.

In step S304, a conversion formula is selected. The selection of the conversion formula is performed by determining the distribution regions 601 to 603 as a processing target on the basis of the clustering information and selecting a conversion formula associated with the distribution regions 601 to 603 as a processing target.

In step S305, a soil moisture amount is estimated. By executing the processing of steps S301 to S304, a soil electrical conductivity in each distribution region is calculated for each of the distribution regions 601 to 603.

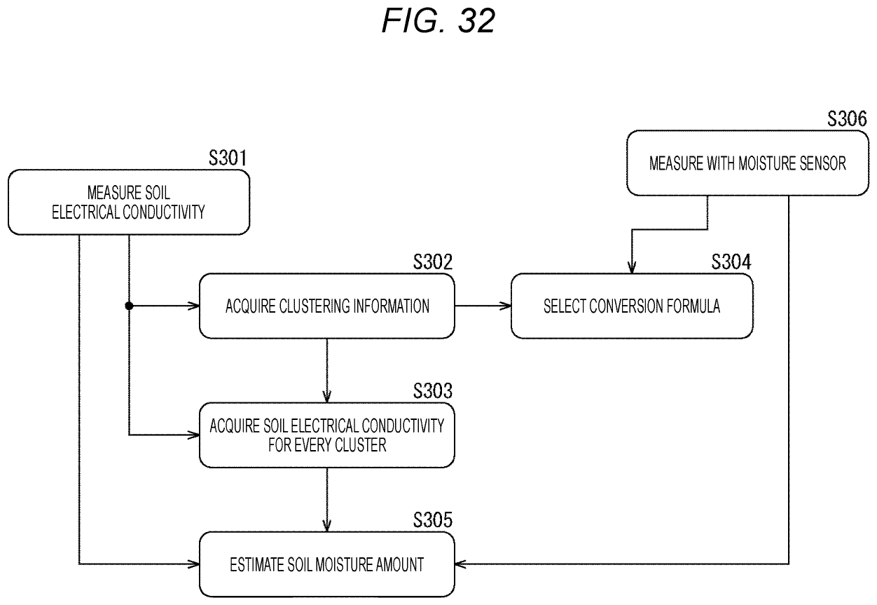

For example, in a case where the distribution region 601 is set as a processing target, the soil electrical conductivity in the distribution region 601 is converted into a soil moisture amount by using the conversion formula (the conversion formula selected in the processing of step S304) associated with the distribution region 601. This conversion is performed as described with reference to FIGS. 28 to 33. By performing such conversion also in the distribution region 602 and the distribution region 603, the moisture amount is estimated for each of the distribution regions 601 to 603.

With the processing so far, the relative soil moisture amount map is generated. The relative soil moisture amount map generated at the time point when the soil electrical conductivity is measured in step S301 can be used as the absolute soil moisture amount map at that time point.

For example, in a case where a device having an automated operation mode is used as the device described above for measuring an electrical conductivity of soil, the measurement of the soil electrical conductivity can be performed in a relatively short cycle. For example, the soil electrical conductivity in the field 400 can be measured (updated) about once a week. In this case, the relative soil moisture amount map is updated about once a week, and update corresponding to a change in soil of the field 400 can be performed.

The processing of executing the processing of steps S301 to S305 to generate the relative soil moisture amount map is performed, for example, on a once-a-month basis or the like.

According to the present embodiment, after the relative soil moisture amount map is generated, information regarding the moisture amount in the field 400 can be acquired substantially in real time, by using the relative soil moisture amount map and the absolute moisture amount data measured by the soil moisture amount sensor 71.

In step S306, the soil moisture amount sensor 71 measures a moisture amount to acquire the absolute moisture amount data 71. The acquired absolute moisture amount data 71 is used to estimate the soil moisture amount in step S305. In this case, processing of reflecting the acquired absolute moisture amount data 71 in the distribution regions 601 to 603 as a processing target is executed using the relative soil moisture amount map. In this way, the absolute soil moisture amount map is created.

A frequency at which the soil moisture amount is measured by the soil moisture amount sensor 71 and the absolute soil moisture amount map is updated can be set to, for example, 24 times per day (every one hour). Therefore, update corresponding to a change in soil of the field 400 can be performed, the update frequency can be increased, and the moisture amount in the field 400 can be grasped more accurately.

Since the moisture amount in the field 400 can be grasped with high accuracy, it is possible to more finely and appropriately perform the processing based on the grasped moisture amount, for example, the irrigation control.

The created absolute soil moisture amount map may be stored, and a state in which the moisture amount changes with the lapse of time may be provided to the user, for example.

### <Recording Medium>

The above-described series of processing can be executed by hardware or software. In a case where the series of processing is performed by software, a program that makes up the software is installed in a computer. Here, examples of the computer include a computer incorporated in dedicated hardware, a computer that can execute various functions by installation of various programs, for example, a general-purpose personal computer, and the like.

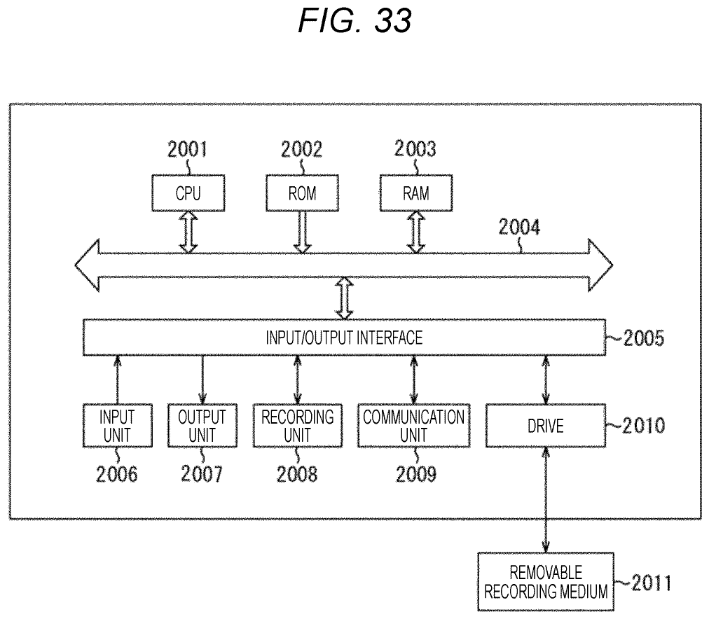

FIG. 33 is a block diagram illustrating a configuration example of hardware of the computer that executes the above-described series of processing by a program. In the computer, a central processing unit (CPU) 2001, a read only memory (ROM) 2002, and a random access memory (RAM) 2003 are connected to one another by a bus 2004. An input/output interface 2005 is further connected to the bus 2004. An input unit 2006, an output unit 2007, a storage unit 2008, a communication unit 2009, and a drive 2010 are connected to the input/output interface 2005.

The input unit 2006 includes a keyboard, a mouse, a microphone, and the like. The output unit 2007 includes a display, a speaker, and the like. The storage unit 2008 includes a hard disk, a nonvolatile memory, and the like. The communication unit 2009 includes a network interface and the like. The drive 2010 drives a removable medium 2011 such as a magnetic disk, an optical disk, a magneto-optical disk, or a semiconductor memory.

In the computer designed as described above, the CPU 2001 loads, for example, a program stored in the storage unit 2008 into the RAM 2003 via the input/output interface 2005 and the bus 2004, and executes the program, so that the series of processing described above is performed.

The program executed by the computer (CPU 2001) can be provided by being recorded in the removable medium 2011 as a package medium or the like, for example. Furthermore, the program can be provided via a wired or wireless transmission medium such as a local area network, the Internet, or digital satellite broadcasting.

In the computer, the program can be installed in the storage unit 2008 via the input/output interface 2005 by mounting the removable medium 2011 on the drive 2010. Furthermore, the program can be received by the communication unit 2009 via a wired or wireless transmission medium and installed in the storage unit 2008. In addition to this, the program can be installed in the ROM 2002 or the storage unit 2008 in advance.

Note that the program to be executed by the computer may be a program that executes processing in time series in accordance with an order described in the present specification, or may be a program that executes processing in parallel or at a necessary timing such as when a call is made.

In the present specification, the system represents the entire device including a plurality of devices.

Note that the effects described in the present specification are merely examples and are not limited, and other effects may be provided.

Note that embodiments of the present technology are not limited to the above-described embodiments, and various changes can be made without departing from the gist of the present technology.

Note that the present technology may also provide the following configurations.

(1)

A data processing apparatus including:
a first creation unit configured to create a relative moisture amount map regarding a relative moisture amount in a field;
an acquisition unit configured to acquire moisture amount data from a moisture amount sensor installed in the field; and
a second creation unit configured to convert the relative moisture amount into an absolute moisture amount by using the moisture amount data, to create an absolute moisture amount map.
   (2)

The data processing apparatus according to (1) above, in which
the first creation unit creates the relative moisture amount map on the basis of a distribution of an electrical conductivity of the field.
   (3)

The data processing apparatus according to (1) or (2) above, in which
the first creation unit creates the relative moisture amount map on the basis of data acquired for the field by an underground Doppler radar.
   (4)

The data processing apparatus according to (1) or (3) above, in which
the second creation unit creates the absolute moisture amount map by adjusting the relative moisture amount in the relative moisture amount map to decrease a difference between the moisture amount data from the moisture amount sensor and data of a relative moisture amount at a location on the relative moisture amount map corresponding to a location where the moisture amount sensor is installed in the field.
   (5) The data processing apparatus according to any one of (1) to (4) above, in which
control of irrigation is performed using the absolute moisture amount map such that a moisture amount in the field becomes uniform.
   (6) The data processing apparatus according to (2) above, in which
the first creation unit
analyzes an image obtained by capturing an image of a crop in the field, and measures the electrical conductivity in accordance with an analysis result, and
updates the relative moisture amount map.
   (7)

The data processing apparatus according to (2) above, in which
the electrical conductivity is acquired in association with location information, by scanning while moving in the field.
   (8)

The data processing apparatus according to (2) above, in which
the electrical conductivity is acquired in association with measurement data acquired for the field by a soil electrical conductivity sensor and location information of the soil electrical conductivity sensor.
   (9)

The data processing apparatus according to any one of (1) to (8) above, in which
the moisture amount sensor is installed at a location in the field corresponding to a barycenter of a region grouped on the basis of a relative moisture amount in the relative moisture amount map.
   (10)

The data processing apparatus according to any one of (1) to (9) above, in which
a larger number of the moisture amount sensors are installed in a region in which a distribution of a relative moisture amount varies in the relative moisture amount map than in a region in which there is no variation.
   (11)

The data processing apparatus according to (2) above, in which
the first creation unit generates a conversion formula for converting the electrical conductivity into a moisture amount, by using the electrical conductivity and the moisture amount data measured in a dry state and the electrical conductivity and the moisture amount data measured in a wet state, and
creates the relative moisture amount map by using the conversion formula.
   (12)

The data processing apparatus according to (11) above, in which
the electrical conductivity in a first region where the moisture amount sensor is installed is calculated on the basis of the electrical conductivity measured in a second region outside the first region.
   (13)

The data processing apparatus according to any one of (1) to (12) above, in which
a water stress of a crop is estimated using the absolute moisture amount map, and a notification is given to a user in a case where the crop is detected in which the water stress is high.
   (14)

A data processing method performed by a data processing apparatus, the data processing method including:
creating a relative moisture amount map regarding a relative moisture amount in a field;
acquiring moisture amount data from a moisture amount sensor installed in the field; and
converting the relative moisture amount into an absolute moisture amount by using the moisture amount data, to create an absolute moisture amount map.
   (15)

A data processing apparatus for
creating a soil moisture amount map indicating a distribution of a soil moisture amount in a field, on the basis of
data related to soil moisture acquired for a first region of the field, and
data of a plurality of soil moisture sensors acquired for a plurality of regions that is included in the first region and is narrower than the first region.
   (16)

The data processing apparatus according to (15) above, in which
the data related to soil moisture includes data acquired by a ground penetrating radar (GPR) or data acquired by a soil electrical conductivity sensor.
   (17)

The data processing apparatus according to (15) or (16) above, in which
by generating an absolute value difference between data of each of the soil moisture sensors and a value obtained by interpolating data related to soil moisture at each installation place of the soil moisture sensors, the soil moisture amount map is generated to minimize a sum of the absolute values.
   (18)

The data processing apparatus according to (17) above, in which
the data processing apparatus outputs data regarding an irrigation condition for minimizing a sum of absolute value differences of soil moisture amounts at a plurality of freely selected points in the soil moisture amount map.
   (19)

A data processing apparatus for generating a first soil moisture amount map on the basis of data related to soil moisture and acquired for a first region of a field, in which
the first soil moisture amount map is generated to include a plurality of areas having relatively different soil moisture amounts.
   (20)

The data processing apparatus according to (19) above, in which
the data related to soil moisture includes data acquired by a ground penetrating radar (GPR) or data acquired by a soil electrical conductivity sensor.
   (21)

The data processing apparatus according to (19) or (20) above, in which
the first soil moisture amount map is generated also on the basis of a soil analysis result of a soil sample sampled from a plurality of points included in the first region.
   (22)

The data processing apparatus according to any one of (19) to (21) above, in which
the first soil moisture amount map is generated to further include a recommended arrangement location of a soil moisture sensor.
   (23) The data processing apparatus according to (19) above, in which
the data related to soil moisture includes data acquired by a soil electrical conductivity sensor, and
the first soil moisture amount map is generated to further include a recommended acquisition location of a soil sample for soil analysis.
   (24) The data processing apparatus according to any one of (19) to (23) above, in which
a second soil moisture amount map having higher accuracy for a soil moisture amount than the first soil moisture map is generated, on the basis of
the first soil moisture map, and
data of a plurality of soil moisture sensors acquired for a plurality of regions that is included in the first region and is narrower than the first region.
   (25)

A data processing apparatus for
calculating a soil moisture amount at a first point of a field, on the basis of measurement data of a soil electrical conductivity sensor acquired for the first point and measurement data of a soil moisture sensor acquired for a second point different from the first point.

### REFERENCE SIGNS LIST

11 Irrigation control system
21 Absolute soil moisture amount measurement system
22 Soil characteristic measurement system
23 Soil electrical conductivity measurement system
24 Relative soil moisture amount map generation system
25 Absolute soil moisture amount estimation system
26 Response processing system
41 Soil sample collection unit
42 Location grasping unit
43 Analysis unit
44 Communication unit
51 Soil electrical conductivity sensor
52 Location grasping unit
53 Sensor conveyance unit
54 Communication unit
61 Data acquisition unit
62 Map generation unit
63 Holding unit
71 Soil moisture sensor
72 Location grasping unit
73 Communication unit
91 Irrigation control unit
92 Alert generation unit
110 Sensor device
120 Sensor head
121 Probe
122 Probe
123 First end portion
124 Second end portion
125 Antenna
130 Measurement unit
131 Directional coupler
132 Transmitter
133 Communication unit
134 Incident wave receiver
135 Reflected wave receiver
136 Transmitted wave receiver
200 Server
202 Probe
331 Region
400 Field
409 Signal
501 Information terminal
503 Display
505 Mark
531 Camera

1. A data processing apparatus comprising:
a first creation unit configured to create a relative moisture amount map regarding a relative moisture amount in a field;
an acquisition unit configured to acquire moisture amount data from a moisture amount sensor installed in the field; and
a second creation unit configured to convert the relative moisture amount into an absolute moisture amount by using the moisture amount data, to create an absolute moisture amount map.

2. The data processing apparatus according to claim 1, wherein
the first creation unit creates the relative moisture amount map on a basis of a distribution of an electrical conductivity of the field.

3. The data processing apparatus according to claim 1, wherein
the first creation unit creates the relative moisture amount map on a basis of data acquired for the field by an underground Doppler radar.

4. The data processing apparatus according to claim 1, wherein
the second creation unit creates the absolute moisture amount map by adjusting the relative moisture amount in the relative moisture amount map to decrease a difference between the moisture amount data from the moisture amount sensor and data of a relative moisture amount at a location on the relative moisture amount map corresponding to a location where the moisture amount sensor is installed in the field.

5. The data processing apparatus according to claim 1, wherein
control of irrigation is performed using the absolute moisture amount map such that a moisture amount in the field becomes uniform.

6. The data processing apparatus according to claim 2, wherein
the first creation unit
analyzes an image obtained by capturing an image of a crop in the field, and measures the electrical conductivity in accordance with an analysis result, and
updates the relative moisture amount map.

7. The data processing apparatus according to claim 2, wherein
the electrical conductivity is acquired in association with location information, by scanning while moving in the field.

8. The data processing apparatus according to claim 2, wherein
the electrical conductivity is acquired in association with measurement data acquired for the field by a soil electrical conductivity sensor and location information of the soil electrical conductivity sensor.

9. The data processing apparatus according to claim 1, wherein
the moisture amount sensor is installed at a location in the field corresponding to a barycenter of a region grouped on a basis of a relative moisture amount in the relative moisture amount map.

10. The data processing apparatus according to claim 1, wherein
a larger number of the moisture amount sensors are installed in a region in which a distribution of a relative moisture amount varies in the relative moisture amount map than in a region in which there is no variation.

11. The data processing apparatus according to claim 2, wherein
the first creation unit generates a conversion formula for converting the electrical conductivity into a moisture amount, by using the electrical conductivity and the moisture amount data measured in a dry state and the electrical conductivity and the moisture amount data measured in a wet state, and
creates the relative moisture amount map by using the conversion formula.

12. The data processing apparatus according to claim 11, wherein
the electrical conductivity in a first region where the moisture amount sensor is installed is calculated on a basis of the electrical conductivity measured in a second region outside the first region.

13. The data processing apparatus according to claim 1, wherein
a water stress of a crop is estimated using the absolute moisture amount map, and a notification is given to a user in a case where the crop is detected in which the water stress is high.

14. A data processing method performed by a data processing apparatus, the data processing method comprising:
creating a relative moisture amount map regarding a relative moisture amount in a field;
acquiring moisture amount data from a moisture amount sensor installed in the field; and
converting the relative moisture amount into an absolute moisture amount by using the moisture amount data, to create an absolute moisture amount map.

15. A data processing apparatus for
creating a soil moisture amount map indicating a distribution of a soil moisture amount in a field, on a basis of
data related to soil moisture acquired for a first region of the field, and
data of a plurality of soil moisture sensors acquired for a plurality of regions that is included in the first region and is narrower than the first region.

16. The data processing apparatus according to claim 15, wherein
the data related to soil moisture includes data acquired by a ground penetrating radar (GPR) or data acquired by a soil electrical conductivity sensor.

17. A data processing apparatus for generating a first soil moisture amount map on a basis of data related to soil moisture and acquired for a first region of a field, wherein
the first soil moisture amount map is generated to include a plurality of areas having relatively different soil moisture amounts.

18. The data processing apparatus according to claim 17, wherein
the first soil moisture amount map is generated to further include a recommended arrangement location of a soil moisture sensor.

19. The data processing apparatus of claim 18, wherein
the data related to soil moisture includes data acquired by a soil electrical conductivity sensor, and
the first soil moisture amount map is generated to further include a recommended acquisition location of a soil sample for soil analysis.

20. A data processing apparatus for
calculating a soil moisture amount at a first point of a field, on a basis of measurement data of a soil electrical conductivity sensor acquired for the first point and measurement data of a soil moisture sensor acquired for a second point different from the first point.
